(19) United States
(12) Patent Application Publication
(10) Pub. No.: US 2021/0251916 A1
(43) Pub. Date: Aug. 19, 2021

(54) TRANSDERMAL THERAPEUTIC SYSTEM CONTAINING RIVASTIGMINE

(71) Applicant: LTS LOHMANN THERAPIE-SYSTEME AG, Andernach (DE)

(72) Inventors: Nico REUM, Mendig (DE); Patrick MOHR, Bad Breisig (DE); Wolfgang LAUX, Diez (DE); Beatrix PLATT, Hausten (DE)

(21) Appl. No.: 17/250,161

(22) PCT Filed: Jun. 18, 2019
(86) PCT No.: PCT/EP2019/066090

§ 371 Date: Dec. 7, 2020

(30) Foreign Application Priority Data
Jun. 19, 2018 (DE) 10 2018 209 916.2

(51) Int. Cl. A61K 9/70; A61K 31/27; A61K 47/32; A61K 47/44; A61F 13/02; B32B 38/04; B32B 7/06; B32B 7/12; B32B 27/36

(52) U.S. Cl. CPC A61K 9/7084; A61K 31/27; A61K 47/32; A61K 47/44; A61F 13/0283; B32B 38/04; B32B 2255/26; B32B 7/12; B32B 27/36; A61F 2013/0296; B32B 2535/00; B32B 2255/10; B32B 7/06

(57) ABSTRACT

The present invention relates to a transdermal therapeutic system for the transdermal administration of rivastigmine comprising a rivastigmine-containing layer structure, said rivastigmine-containing layer structure comprising: A) a backing layer; B) a rivastigmine-containing layer comprising at least one acrylic polymer; and C) a skin contact layer comprising at least one styrene-isoprene-styrene block copolymer and at least one tackifier.

### TECHNICAL FIELD OF THE INVENTION

The present invention relates to a transdermal therapeutic system (TTS) for the transdermal administration of rivastigmine to the systemic circulation, and processes of manufacture, method of treatments and uses thereof.

### BACKGROUND OF THE INVENTION

The active agent rivastigmine (also known as (S)-3-[1-(Dimethylamino)ethyl]phenyl ethylmethylcarbamate, CAS Number 123441-03-2) is an parasympathomimetic or cholinergic agent belonging to the family of phenyl carbamate. It has the following chemical formula.

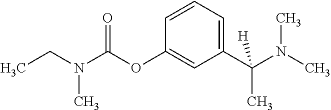 [SMILES: CCN(C)C(=O)Oc1cccc([C@H](C)N(C)C)c1]

Rivastigmine inhibits both butyrylcholinesterase and acetylcholinesterase. In general, rivastigmine is used for the treatment of mild to moderate dementia of the Alzheimer's type and dementia due to Parkinson's disease.

Currently, rivastigmine is commercially available, e.g., in the form of capsules and in the form of transdermal therapeutic systems.

A transdermal therapeutic system, which is commercially available under the name Exelon® has an area of release of 2.5, 5, 7.5 10, 15, or 20 cm². According to EP 2 292 219 B2, the TTS comprises three layers in the following order: (1) a backing layer, (2) a rivastigmine-containing layer comprising acrylate polymers, and (3) an adhesive layer free of rivastigmine comprising a silicone adhesive.

Exelon® comprises, depending on the patch size, 4.5, 9, 13.5, 18, 27, or 36 mg of rivastigmine. The TTS is designed to deliver approximately 2.3, 4.6, 6.7, 9.5, 13.3, or 17.4 mg of rivastigmine over a 24-hour period.

One problem in connection with Exelon® is that the currently available patches tend to cause skin irritation, in particular in certain patient groups, e.g. Japanese patients.

It is therefore desirable to provide a TTS, which causes less skin irritation in comparison to Exelon®.

There is thus a need in the art for an improved transdermal therapeutic system for the transdermal administration of rivastigmine.

### OBJECTS AND SUMMARY OF THE INVENTION

It is an object of the present invention to provide a TTS for the transdermal administration of rivastigmine, which is improved in comparison to the current commercially available rivastigmine TTS Exelon®.

It is a further object of the present invention to provide a TTS for the transdermal administration of rivastigmine without causing significant skin irritation problems.

It is a further object of the present invention to provide a TTS for the transdermal administration of rivastigmine, which is suitable for use in a method of preventing, treating, or delaying of progression of Alzheimer's disease, dementia associated with Parkinson's disease, and/or symptoms of traumatic brain injury, or for use in a method of treating mild to moderate dementia caused by Alzheimer's or Parkinson's disease.

It has now surprisingly been found that at least one of these objects and others are accomplished by the present invention, which according to one aspect relates to a transdermal therapeutic system for the transdermal administration of rivastigmine comprising a rivastigmine-containing layer structure, said rivastigmine-containing layer structure comprising:
A) a backing layer;
B) a rivastigmine-containing layer comprising at least one acrylic polymer; and
C) a skin contact layer comprising at least one styrene-isoprene-styrene block copolymer and at least one tackifier.
   In particular, it has been found that due to the fact that the TTS comprises an active-containing acrylic layer as well as an additional skin contact layer, wherein preferably the at least one styrene-isoprene-styrene block copolymer and the at least one tackifier are present in the skin contact layer in an overall amount of at least 90% by weight, more preferably in an overall amount of at least 99% by weight based on the total weight of the skin contact layer, results in permeation properties comparable to the market product Exelon®, while at the same time providing very good adhesive properties. In particular, the skin contact layer surprisingly does not have a negative impact on the release properties of the TTS. The invention also relates to a process for manufacturing a transdermal therapeutic system comprising the steps of:
1) providing a rivastigmine-containing coating composition by combining at least the components
   i) rivastigmine; and
   ii) at least one acrylic polymer;
2) coating the rivastigmine-containing coating composition onto a film in an amount to provide the desired area weight,
3) drying the coated rivastigmine-containing coating composition to provide the rivastigmine-containing layer,
4) providing an additional coating composition for an additional skin contact layer by combining at least the components
   a) at least one styrene-isoprene-styrene block copolymer; and
   b) at least one tackifier;
5) coating and drying the additional coating composition according to steps 2 and 3, wherein the film is a release liner,
6) laminating the adhesive side of the skin contact layer onto the adhesive side of the rivastigmine-containing layer to provide a rivastigmine-containing layer structure with the desired area of release,
7) punching the individual systems from the rivastigmine-containing layer structure,
8) optionally adhering to the individual systems a rivastigmine-free self-adhesive layer structure comprising also a backing layer and a rivastigmine-free pressure-sensitive adhesive layer and which is larger than the individual systems of rivastigmine-containing self-adhesive layer structure.

### DEFINITIONS

Within the meaning of this invention, the term “transdermal therapeutic system” (TTS) refers to a system by which the active agent (e.g. rivastigmine) is administered to the systemic circulation via transdermal delivery and refers to the entire individual dosing unit that is applied, after removing an optionally present release liner, to the skin of a patient, and which comprises a therapeutically effective amount of active agent in an active agent-containing layer structure and optionally an additional adhesive overlay on top of the active agent-containing layer structure. The active agent-containing layer structure may be located on a release liner (a detachable protective layer), thus, the TTS may further comprise a release liner. Within the meaning of this invention, the term “TTS” in particular refers to systems providing transdermal delivery, excluding active delivery for example via iontophoresis or microporation. Transdermal therapeutic systems may also be referred to as transdermal drug delivery systems (TDDS) or transdermal delivery systems (TDS).

Within the meaning of this invention, the term “rivastigmine-containing layer structure” refers to the layer structure containing a therapeutically effective amount of rivastigmine and comprises a backing layer, a rivastigmine-containing layer and a skin contact layer.

Within the meaning of this invention, the term “therapeutically effective amount” refers to a quantity of active agent in the TTS sufficient to provide, if administered by the TTS to a patient, prevents, treats, or delays of progression of Alzheimer's disease, dementia associated with Parkinson's disease, and/or symptoms of traumatic brain injury. A TTS usually contains more active in the system than is in fact provided to the skin and the systemic circulation. This excess amount of active agent is usually necessary to provide enough driving force for the delivery from the TTS to the systemic circulation.

Within the meaning of this invention, the terms “active”, “active agent”, and the like, as well as the term “rivastigmine” refer to rivastigmine in any pharmaceutically acceptable chemical and morphological form and physical state. These forms include without limitation rivastigmine in its free base/free acid form, protonated or partially protonated rivastigmine, rivastigmine salts, cocrystals and in particular acid/base addition salts formed by addition of an inorganic or organic acid/base such as rivastigmine hydrochloride or rivastigmine tartrate, solvates, hydrates, clathrates, complexes and so on, as well as rivastigmine in the form of particles which may be micronized, crystalline and/or amorphous, and any mixtures of the aforementioned forms. The rivastigmine, where contained in a medium such as a solvent, may be dissolved or dispersed or in part dissolved and in part dispersed.

When rivastigmine is mentioned to be used in a particular form in the manufacture of the TTS, this does not exclude interactions between this form of rivastigmine and other ingredients of the rivastigmine-containing layer structure, e.g. salt formation or complexation, in the final TTS. This means that, even if rivastigmine is included in its free base/acid form, it may be present in the final TTS in protonated or partially protonated/or deprotonated or partially deprotonated form or in the form of an acid addition salt, or, if it is included in the form of a salt, parts of it may be present as free base in the final TTS. Unless otherwise indicated, in particular the amount of rivastigmine in the layer structure relates to the amount of rivastigmine included in the TTS during manufacture of the TTS and is calculated based on rivastigmine in the form of the free base. E.g., when a) 0.1 mmol (equal to 25.03 mg) rivastigmine base or b) 0.1 mmol (equal to 40.04 mg) rivastigmine tartrate is included in the TTS during manufacture, the amount of rivastigmine in the layer structure is, within the meaning of the invention, in both cases 0.1 mmol or 25.03 mg.

The rivastigmine starting material included in the TTS during manufacture of the TTS may be in the form of particles. Rivastigmine may e.g. be present in the rivastigmine-containing layer structure in the form of particles and/or dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “dispersing” refers to a step or a combination of steps wherein a starting material (e.g. rivastigmine) is not totally dissolved. Dispersing in the sense of the invention comprises the dissolution of a part of the starting material (e.g. rivastigmine particles), depending on the solubility of the starting material (e.g. the solubility of rivastigmine in the coating composition).

There are two main types of TTS for active agent delivery, i.e. matrix-type TTS and reservoir-type TTS. The release of the active agent in a matrix-type TTS is mainly controlled by the matrix including the active agent itself. In contrast thereto, a reservoir-type TTS typically needs a rate-controlling membrane controlling the release of the active agent. In principle, also a matrix-type TTS may contain a rate-controlling membrane. However, matrix-type TTS are advantageous in that, compared to reservoir-type TTS, usually no rate determining membranes are necessary and no dose dumping can occur due to membrane rupture. In summary, matrix-type transdermal therapeutic systems (TTS) are less complex in manufacture and easy and convenient to use by patients.

Within the meaning of this invention, “matrix-type TTS” refers to a system or structure wherein the active is homogeneously dissolved and/or dispersed within a polymeric carrier, i.e. the matrix, which forms with the active agent and optionally remaining ingredients a matrix layer. In such a system, the matrix layer controls the release of the active agent from the TTS. Preferably, the matrix layer has sufficient cohesion to be self-supporting so that no sealing between other layers is required. Accordingly, the active agent-containing layer may in one embodiment of the invention be an active agent-containing matrix layer, wherein the active agent is homogeneously distributed within a polymer matrix. In certain embodiments, the active agent-containing matrix layer may comprise two active agent-containing matrix layers, which may be laminated together. Matrix-type TTS may in particular be in the form of a “drug-in-adhesive”-type TTS referring to a system wherein the active is homogeneously dissolved and/or dispersed within a pressure-sensitive adhesive matrix. In this connection, the active agent-containing matrix layer may also be referred to as active agent-containing pressure sensitive adhesive layer or active agent-containing pressure sensitive adhesive matrix layer. A TTS comprising the active agent dissolved and/or dispersed within a polymeric gel, e.g. a hydrogel, is also considered to be of matrix-type in accordance with present invention.

TTS with a liquid active agent-containing reservoir are referred to by the term “reservoir-type TTS”. In such a system, the release of the active agent is preferably controlled by a rate-controlling membrane. In particular, the reservoir is sealed between the backing layer and the rate-controlling membrane. Accordingly, the active agent-containing layer may in one embodiment be an active agent-containing reservoir layer, which preferably comprises a liquid reservoir comprising the active agent. Furthermore, the reservoir-type TTS typically additionally comprises a skin contact layer, wherein the reservoir layer and the skin contact layer may be separated by the rate-controlling membrane. In the reservoir layer, the active agent is preferably dissolved in a solvent such as ethanol or water or in silicone oil. The skin contact layer typically has adhesive properties.

Reservoir-type TTS are not to be understood as being of matrix-type within the meaning of the invention. However, microreservoir TTS (biphasic systems having deposits (e.g. spheres, droplets) of an inner active-containing phase dispersed in an outer polymer phase), considered in the art to be a mixed form of a matrix-type TTS and a reservoir-type TTS that differ from a homogeneous single phase matrix-type TTS and a reservoir-type TTS in the concept of drug transport and drug delivery, are considered to be of matrix-type within the meaning of the invention. The sizes of microreservoir droplets can be determined by an optical microscopic measurement (for example by Leica MZ16 including a camera, for example Leica DSC320) by taking pictures of the microreservoirs at different positions at an enhancement factor between 10 and 400 times, depending on the required limit of detection. By using imaging analysis software, the sizes of the microreservoirs can be determined.

Within the meaning of this invention, the term “rivastigmine-containing layer” refers to a layer containing the active agent rivastigmine and providing the area of release. The term covers rivastigmine-containing matrix layers and rivastigmine-containing reservoir layers. If the rivastigmine-containing layer is a rivastigmine-containing matrix layer, said layer is present in a matrix-type TTS. The additional skin contact layer is present as adhesive layer, and an adhesive overlay may be provided. The additional skin contact layer is typically manufactured such that it is active agent-free. However, due to the concentration gradient, the active agent rivastigmine will migrate from the matrix layer to the additional skin contact layer over time, until equilibrium is reached. The additional skin contact layer may be present on the rivastigmine-containing matrix layer or separated from the rivastigmine-containing matrix layer by a membrane, preferably a rate controlling membrane. If the rivastigmine-containing layer is a rivastigmine-containing reservoir layer, said layer is present in a reservoir-type TTS, and the layer comprises the active agent rivastigmine in a liquid reservoir. In addition, an additional skin contact layer is present in order to provide adhesive properties. Preferably, a rate-controlling membrane separates the reservoir layer from the additional skin contact layer. The additional skin contact layer can be manufactured such that it is active agent-free or active agent-containing. If the additional skin contact layer is free of active agent the active agent will migrate, due to the concentration gradient, from the reservoir layer to the skin contact layer over time, until equilibrium is reached. Additionally an adhesive overlay may be provided.

As used herein, the rivastigmine-containing layer is preferably a rivastigmine-containing matrix layer, and it is referred to the final solidified layer. Preferably, a rivastigmine-containing matrix layer is obtained after coating and drying the solvent-containing coating composition as described herein. Alternatively a rivastigmine-containing matrix layer is obtained after melt-coating and cooling. The rivastigmine-containing matrix layer may also be manufactured by laminating two or more such solidified layers (e.g. dried or cooled layers) of the same composition to provide the desired area weight. The matrix layer may be self-adhesive (in the form of a pressure sensitive adhesive matrix layer). The TTS comprises an additional skin contact layer for providing sufficient tack. Preferably, the matrix layer is a pressure sensitive adhesive matrix layer. Optionally, an adhesive overlay may be present.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. A pressure sensitive adhesive layer, when in contact with the skin, is “self-adhesive”, i.e. provides adhesion to the skin so that typically no further aid for fixation on the skin is needed. A “self-adhesive” layer structure includes a pressure sensitive adhesive layer for skin contact which may be provided in the form of a pressure sensitive adhesive matrix layer or in the form of an additional layer, i.e. a pressure sensitive adhesive skin contact layer. An adhesive overlay may still be employed to advance adhesion. The pressure-sensitive adhesive properties of a pressure-sensitive adhesive depend on the polymer or polymer composition used.

As used herein, an rivastigmine-containing matrix layer is a layer containing the active agent rivastigmine dissolved or dispersed in at least one acrylic polymer, or containing the active agent rivastigmine dissolved in a solvent to form a rivastigmine-solvent mixture that is dispersed in the form of deposits (in particular droplets) in at least one acrylic polymer. Preferably, the at least one acrylic polymer is an acrylic pressure-sensitive adhesive. Within the meaning of this invention, the term “pressure-sensitive adhesive layer” refers to a pressure-sensitive adhesive layer obtained from a solvent-containing adhesive coating composition after coating on a film and evaporating the solvents.

Within the meaning of this invention, the term “skin contact layer” refers to the layer included in the rivastigmine-containing layer structure to be in direct contact with the skin of the patient during administration. In a TTS comprising an additional skin contact layer, the other layers of the rivastigmine-containing layer structure do not contact the skin and do not necessarily have self-adhesive properties. As outlined above, an additional skin contact layer attached to the rivastigmine-containing layer may over time absorb parts of the rivastigmine. An additional skin contact layer may be used to enhance adherence. The sizes of an additional skin contact layer and the rivastigmine-containing layer are usually coextensive and correspond to the area of release. However, the area of the additional skin contact layer may also be greater than the area of the rivastigmine-containing layer. In such a case, the area of release still refers to the area of the rivastigmine-containing layer.

Within the meaning of this invention, the term “area weight” refers to the dry weight of a specific layer, e.g. of the matrix layer, provided in g/m². The area weight values are subject to a tolerance of ±10%, preferably ±7.5%, due to manufacturing variability.

If not indicated otherwise “%” refers to % by weight.

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, preferably above 5000 and more preferably above 10,000 Dalton. Correspondingly, compounds with a molecular weight below 2000, preferably below 5000 or more preferably below 10,000 Dalton are usually referred to as oligomers.

Within the meaning of this invention, the term “adhesive overlay” refers to a self-adhesive layer structure that is free of active agent and larger in area than the rivastigmine-containing structure and provides additional area adhering to the skin, but no area of release of the active agent rivastigmine. It enhances thereby the overall adhesive properties of the TTS. The adhesive overlay comprises a backing layer that may provide occlusive or non-occlusive properties and an adhesive layer. Preferably, the backing layer of the adhesive overlay provides non-occlusive properties.

Within the meaning of this invention, the term “backing layer” refers to a layer which supports the rivastigmine-containing layer or forms the backing of the adhesive overlay. At least one backing layer in the TTS and usually the backing layer of the rivastigmine-containing layer is substantially impermeable to the active agent rivastigmine contained in the layer during the period of storage and administration and thus prevents active loss or cross-contamination in accordance with regulatory requirements. Preferably, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyurethanes, and mixtures thereof. Suitable backing layers are thus for example PET laminates, EVA-PET laminates and PE-PET laminates. Also suitable are woven or non-woven backing materials.

The TTS according to the present invention can be characterized by certain parameters as measured in an in vitro release test.

Within the meaning of this invention the “in vitro release rate” is determined using a rotating cylinder apparatus of the Ph Eur/USP using 600 ml degassed 0.9% sodium chloride solution at 32° C. and rotated at 50 rpm. At 0.5, 2, 4, 7 and 24 hours, 4 ml samples are removed and analyzed using a validated HPLC method with a UV photometric detector.

The TTS according to the present invention can also be characterized by certain parameters as measured in an in vitro skin permeation test.

In general, the in vitro permeation test is performed in a Franz diffusion cell, with EVA membrane (e.g. 9% vinyl acetate), and with phosphate buffer pH 5.5 or 7.4 as receptor medium (32° C. with 0.1% saline azide).

Further, in vitro permeation tests may be performed in a Franz diffusion cell, with human or animal skin and preferably with dermatomed split-thickness human skin with a thickness of 800 um and an intact epidermis, and with phosphate buffer pH 5.5 or 7.4 as receptor medium (32° C. with 0.1% saline azide) with or without addition of a maximum of 40 vol-% organic solvent e.g. ethanol, acetonitrile, isopropanol, dipropylenglycol, PEG 400 so that a receptor medium may e.g. contain 60 vol-% phosphate buffer pH 5.5, 30 vol-% dipropylenglycol and 10 vol-% acetonitrile.

Where not otherwise indicated, the in vitro permeation test is performed with EVA membrane (9% vinyl acetate, 50 gm), and with phosphate buffer pH 5.5 as receptor medium (32° C. with 0.1% saline azide). The amount of active agent permeated into the receptor medium is determined in regular intervals using a validated HPLC method (column: stainless steel column 150 mm x 3.9 mm I.D. packed with C18-Phase (e.g. Novapak C18, 4gm particle size, Waters) or equivalent column, column temperature: 20-25° C.; mobile phase: Acetonitrile/Water 20:80 (v/v)+0.35mL TEA per 100mL, pH 3.5; adjust the pH with phosphoric acid (85%), if necessary; flow rate: 1.0 ml/min; pressure: approx. 100 bar; injection volume: 20 gL; Detection: UV, 210 nm, stop time: 6 min). The receptor medium is completely or in part replaced by fresh medium when taking the sample volume, and the measured amount of active agent permeated relates to the amount permeated between the two last sampling points and not the total amount permeated so far.

Thus, within the meaning of this invention, the parameter “permeated amount” is provided in μg/cm² and relates to the amount of active agent permeated in a sample interval at certain elapsed time. E.g., in an in vitro permeation test as described above, wherein the amount of active agent permeated into the receptor medium has been e.g. measured at hours 0, 2, 4, 8, 12 and 24, the “permeated amount” of active agent can be given e.g. for the sample interval from hour 8 to hour 12 and corresponds to the measurement at hour 12, wherein the receptor medium has been exchanged completely at hour 8.

The permeated amount can also be given as a “cumulative permeated amount”, corresponding to the cumulated amount of active agent permeated at a certain point in time. E.g., in an in vitro permeation test as described above, wherein the amount of active agent permeated into the receptor medium has been e.g. measured at hours 0, 2, 4, 8, 12 and 24, the “cumulative permeated amount” of active agent at hour 12 corresponds to the sum of the permeated amounts from hour 0 to hour 2, hour 2 to hour 4, hour 4 to hour 8 and hour 8 to hour 12.

Within the meaning of this invention, the parameter “skin permeation rate” for a certain sample interval at certain elapsed time is provided in μg/cm²-hr and is calculated from the permeated amount in said sample interval as measured by in vitro permeation test as described above in μg/cm², divided by the hours of said sample interval. E.g. the skin permeation rate in an in vitro permeation test as described above, wherein the amount of active agent permeated into the receptor medium has been e.g. measured at hours 0, 2, 4, 8, 12 and 24, the “skin permeation rate” at hour 12 is calculated as the permeated amount in the sample interval from hour 8 to hour 12 divided by 4 hours.

A “cumulative skin permeation rate” can be calculated from the respective cumulative permeated amount by dividing the cumulative permeated amount by the elapsed time. E.g. in an in vitro permeation test as described above, wherein the amount of active agent permeated into the receptor medium has been e.g. measured at hours 0, 2, 4, 8, 12 and 24, the “cumulative skin permeation rate” at hour 12 is calculated as the cumulative permeated amount for hour 12 (see above) divided by 12 hours.

Within the meaning of this invention, the above parameters “permeated amount” and “skin permeation rate” (as well as “cumulative permeated amount” and “cumulative skin permeation rate”) refer to mean values calculated from at least 3 in vitro permeation test experiments. Where not otherwise indicated, the standard deviation (SD) of these mean values refer to a corrected sample standard deviation, calculated using the formula:

$$S D = \sqrt{\frac{1}{n - 1} {{\sum }_{i = 1}}^{n} {( {x}_{i} - \overline{x} )}^{2}}$$

wherein n is the sample size, {x₁, x₂, . . . xₙ} are the observed values and x̅ is the mean value of the observed values.

The TTS according to the present invention can also be characterized by certain parameters as measured in an in vivo clinical study.

Within the meaning of this invention, the parameter “mean release rate” refers to the mean release rate in μg/hr or in mg/day over the period of administration (e.g., 1 to 7 days) by which the active agent is released through the human skin into the systemic circulation and is based on the AUC obtained over said period of administration in a clinical study.

Within the meaning of this invention, the term “extended period of time” relates to a period of at least about 24 h, or at least about 48 h, or at least about 84 h, or at least about 168 h, or at least about 1 day, or at least about 3.5 days, or at least or about 7 days, or to a period of about 24 h to about 168 h or 1 to 7 day(s), or about 24 h to about 84 h or 1 to 3.5 day(s).

For a continuous drug treatment, the frequency of drug administration is preferably kept sufficiently high so as to maintain a therapeutically effective blood plasma concentration. In other words, the interval between two dosage form administrations, also called dosing interval, needs to be adapted accordingly. Within the meaning of the present invention, the term “dosing interval” refers to the period of time between two consecutive TTS administrations, i.e. the interval between two consecutive points in time a TTS is applied to the skin of the patient. Once applied, the TTS is usually maintained on the skin of the patient for the entire dosing interval and only removed at the end of the dosing interval, at which time a new TTS is applied to the skin. E.g., if the dosing interval is 24 hours or 1 day, the TTS is applied to and maintained on the skin of the patient for 24 hours or 1 day. After 24 hours or 1 day, the TTS is removed from the skin and a new TTS is applied. Thus, a dosing interval of 24 hours or 1 day allows a daily TTS exchange mode in an around-the-clock treatment.

Within the meaning of this invention, the term “room temperature” refers to the unmodified temperature found indoors in the laboratory where the experiments are conducted and usually lies within 15 to 35° C., preferably within 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

Within the meaning of this invention, the term “coating composition” refers to a composition comprising all components of the matrix layer in a solvent, which may be coated onto the backing layer or release liner to form the matrix layer upon drying.

Within the meaning of this invention, the term “pressure sensitive adhesive composition” refers to a pressure sensitive adhesive at least in mixture with a solvent (e.g. n-heptane or ethyl acetate).

Within the meaning of this invention, the term “dissolve” refers to the process of obtaining a solution, which is clear and does not contain any particles, as visible to the naked eye.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, toluene and mixtures thereof.

### BRIEF DESCRIPTION OF THE DRAWINGS

FIG. 1 depicts the cumulative rivastigmine in vitro permeation over 24 hours from Comparative Example 1 and Examples 1 and 3 to 5 after production (“initial”).

FIG. 2 depicts the adhesion force of Comparative Example 1 and Examples 2 to 5 after production (“initial”).

FIG. 3 depicts the peel force of Comparative Example 1 and Examples 1 to 5 after production (“initial”).

FIG. 4 depicts the rivastigmine in vitro release rate over 24 hours from Comparative

Example 1 and Examples 1 to 5 after production (“initial”).

### DETAILED DESCRIPTION

### TTS Structure

The present invention relates to a transdermal therapeutic system for the transdermal administration of rivastigmine comprising a rivastigmine-containing layer structure, said rivastigmine-containing layer structure comprising:
A) a backing layer;
B) a rivastigmine-containing layer comprising at least one acrylic polymer; and
C) a skin contact layer comprising at least one styrene-isoprene-styrene block copolymer and at least one tackifier.

The TTS according to the present invention may be a matrix-type TTS or a reservoir-type TTS, and preferably is a matrix-type TTS.

In a matrix-type TTS according to the invention, the rivastigmine is homogeneously dissolved and/or dispersed within a polymeric carrier, i.e. the matrix, which forms with the rivastigmine and optionally remaining ingredients a matrix layer. Accordingly, the rivastigmine-containing layer may in one embodiment of the invention be a rivastigmine-containing matrix layer, wherein the rivastigmine is homogeneously distributed within a polymer matrix. If a rivastigmine-containing matrix layer is prepared by laminating together two rivastigmine-containing matrix layers, which are of substantially the same composition, the resulting double layer is to be regarded as one rivastigmine-containing matrix layer.

In a reservoir-type TTS according to the present invention, the rivastigmine-containing layer is a rivastigmine-containing reservoir layer, which preferably comprises a liquid reservoir comprising the rivastigmine. The reservoir-type TTS additionally comprises a skin contact layer, wherein the reservoir layer and the skin contact layer are preferably separated by the rate-controlling membrane. Preferably, the skin contact layer is manufactured such that it is rivastigmine-free.

The rivastigmine-containing layer structure is preferably a rivastigmine-containing self-adhesive layer structure. It is preferred that the rivastigmine-containing layer, which is preferably a rivastigmine-containing matrix layer, is self-adhesive. Thus, in a preferred embodiment, the rivastigmine-containing layer structure is a rivastigmine-containing self-adhesive layer structure. Alternatively or additionally, it is preferred that the rivastigmine-containing layer is directly attached to the backing layer, so that there is no additional layer between the backing layer and the rivastigmine-containing layer. Consequently, a layer structure of low complexity is obtained, which is advantageous, e.g., in terms of the costs for the manufacture.

In particular, it is preferred that the rivastigmine-containing layer structure comprises not more than three layers. Sufficient adhesion between the rivastigmine-containing self-adhesive layer structure and the skin of the patient during administration is provided by the skin contact layer.

It is to be understood that the TTS according to the invention contains a therapeutically effective amount of rivastigmine. Thus, in a preferred embodiment of the invention, the rivastigmine-containing layer structure contains a therapeutically effective amount of rivastigmine. The rivastigmine in the rivastigmine-containing layer structure is preferably present in the form of the free base. Preferred embodiments regarding the rivastigmine in the TTS according to the invention are provided further below.

It is preferred according to the invention that the area of release of the TTS is rather small. According to one specific embodiment of the invention, the area of release ranges from 1 to 30 cm², preferably from 2 to 22 cm².

In a preferred embodiment of the invention, the backing layer is substantially rivastigmine impermeable. Furthermore, it is preferred that the backing layer is occlusive.

According to certain embodiments of the invention, the TTS may further comprise an adhesive overlay. This adhesive overlay is in particular larger in area than the rivastigmine-containing structure and is attached thereto for enhancing the adhesive properties of the overall transdermal therapeutic system. Said adhesive overlay comprises a backing layer and an adhesive layer. The adhesive overlay provides additional area adhering to the skin but does not add to the area of release of the rivastigmine. The adhesive overlay comprises a self-adhesive polymer or a self-adhesive polymer mixture selected from the group consisting of silicone acrylic hybrid polymers, acrylic polymers, polysiloxanes, polyisobutylenes, styrene-isoprene-styrene copolymers, and mixtures thereof, which may be identical to or different from any polymer or polymer mixture included in the rivastigmine-containing layer structure.

The rivastigmine-containing layer structure according to the invention, such as a rivastigmine-containing self-adhesive layer structure, is normally located on a detachable protective layer (release liner), from which it is removed immediately before application to the surface of the patient's skin. Thus, the TTS may further comprise a release liner. A TTS protected this way is usually stored in a blister pack or a seam-sealed pouch. The packaging may be child resistant and/or senior friendly.

### Rivastigmine-Containing Layer

As outlined in more detail above, the TTS according to the present invention comprises a rivastigmine-containing layer structure comprising a rivastigmine-containing layer. Preferably, the rivastigmine-containing layer structure is a rivastigmine-containing self-adhesive layer structure. Accordingly, it is also preferred that the rivastigmine-containing layer is a self-adhesive rivastigmine-containing layer, more preferably a self-adhesive rivastigmine-containing matrix layer. In a preferred embodiment, the rivastigmine-containing layer comprises a therapeutically affective amount of the rivastigmine.

In one embodiment of the invention, the rivastigmine-containing layer is a rivastigmine-containing matrix layer. In another embodiment, the rivastigmine-containing layer is a rivastigmine-containing reservoir layer. It is preferred that the rivastigmine-containing layer is a rivastigmine-containing matrix layer.

The rivastigmine-containing layer comprises:
1. rivastigmine, preferably in the form of the free base; and
2. at least one acrylic polymer.

In a preferred embodiment, the rivastigmine-containing layer is a rivastigmine-containing matrix layer comprising
1. rivastigmine, preferably in the form of the free base; and
2. at least one acrylic polymer.

In a preferred embodiment, the invention relates to a rivastigmine-containing layer structure, wherein the at least one acrylic polymer is an acrylic pressure-sensitive adhesive.

In one embodiment of the invention, the rivastigmine-containing layer is obtainable by dissolving, dispersing, or partly dissolving and partly dispersing the rivastigmine, preferably in the form of the free base. As a result, the rivastigmine-containing layer of the TTS according to the invention typically comprises rivastigmine in the form of the free base. In addition, the rivastigmine may, in certain embodiments of the invention, partly be present in protonated form. However, it is preferred that at least 50 mol %, preferably at least 75 mol % of the rivastigmine in the rivastigmine-containing layer are present in the form of the free base. In a particular preferred embodiment, at least 90 mol %, preferably at least 95 mol %, more preferably at least 99 mol % of the rivastigmine in the rivastigmine-containing layer are present in the form of the free base.

In one embodiment of the invention, the amount of rivastigmine contained in the rivastigmine-containing layer structure ranges from 0.5 to 5 mg/cm², preferably from 1 to 3 mg/cm².

In one embodiment of the invention, the rivastigmine-containing layer comprises rivastigmine in an amount of from 20 to 40% by weight, preferably from 25 to 35% by weight, most preferably in an amount of 30% by weight, based on the total weight of the rivastigmine-containing layer.

In one embodiment, the rivastigmine-containing matrix layer composition may comprise a second polymer or may comprise two or more further polymers.

It is to be understood that the TTS according to the present invention may also comprise one or more polymers in addition to the at least one acrylic polymer. Exemplarily, polymers based on polysiloxanes, acrylates, polyisobutylenes, or styrene-isoprene-styrene block copolymers may be used. In one embodiment of the invention, the additional polymer is a pressure-sensitive adhesive based on polysiloxanes, acrylates, or polyisobutylene. Additional polymers may also be added to enhance cohesion and/or adhesion. In yet another preferred embodiment, the invention relates to a transdermal therapeutic system, wherein the rivastigmine-containing layer does not comprise a permeation enhancer or solubilizer.

In certain embodiments of the invention, the acrylic polymer is contained in the rivastigmine-containing layer in an amount of from 5 to 40%, preferably from 8 to 35% by weight based on the total weight of the rivastigmine-containing layer.

In a preferred embodiment of the invention, the acrylic polymer in the rivastigmine-containing layer does not comprise OH-groups as functional groups. Instead, it is preferred that the acrylic polymer in the rivastigmine-containing layer is a COOH-functionalized acrylic polymer, preferably a COOH-functionalized acrylic polymer obtainable from one or more monomers selected from acrylic acid, 2-ethylhexylacrylate, glycidylmethacrylate and methylacrylate. Particularly preferably, the acrylic polymer in the rivastigmine-containing layer is the acrylate-based pressure-sensitive adhesive Duro-Tak™ 384-2353, a copolymer based on acrylic acid, 2-ethylhexylacrylate, glycidylmethacrylate and methylacrylate, provided as a solution in ethyl acetate and hexane.

In one embodiment of the invention, the area weight of the rivastigmine-containing layer ranges from 40 to 250 g/m², preferably from 50 to 200 g/m². In certain preferred embodiments, the area weight ranges from 60 to 180 g/m².

### Skin Contact Layer

As outlined in more detail above, the agent-containing layer structure of the TTS according to the present invention comprises a backing layer, a rivastigmine-containing layer, and a skin contact layer. The skin contact layer is preferably in contact with the rivastigmine-containing layer.

The skin contact layer comprises at least one styrene-isoprene-styrene block copolymer. In particular, the at least one styrene-isoprene-styrene block copolymer is a pressure-sensitive adhesive based on styrene-isoprene-styrene block copolymers. Further details regarding the polymers according to the invention are provided further below.

In certain preferred embodiments, the at least one styrene-isoprene-styrene block copolymer is comprised in the skin contact layer in an amount of from about 20% to about 90%, preferably of from about 30% to about 80%, or of from about 40% to about 60% by weight based on the total weight of the skin contact layer.

The skin contact layer comprises at least one tackifier. In a preferred embodiment, the at least one tackifier is an alicyclic saturated hydrocarbon resin, or a hydrogenated rosin glycerol ester, or paraffinum liquidum, or a mixture thereof. Further details regarding the tackifiers according to the invention are provided further below.

In certain preferred embodiments, the at least one tackifier is comprised in the skin contact layer in an amount of from about 20 to about 80%, preferably from about 35 to about 65%.

In a preferred embodiment, the at least one styrene-isoprene-styrene block copolymer and the at least one tackifier are comprised in the skin contact layer in a ratio of from about 60:40 (w/w) to about 40:60 (w/w), preferably in a ratio of about 50:50 (w/w) based on the total weight of the skin contact layer.

In a particularly preferred embodiment, the at least one styrene-isoprene-styrene block copolymer and the at least one tackifier are comprised in the skin contact layer in a ratio of from about 60:40 (w/w) to about 40:60 (w/w).

In another preferred embodiment, the at least one styrene-isoprene-styrene block copolymer and the at least one tackifier are present in the skin contact layer in an overall amount of at least 90% by weight, preferably in an overall amount of at least 99% by weight based on the total weight of the skin contact layer. Preferably, the skin contact layer does not comprise an acrylic polymer.

The skin contact layer may comprise an active agent. In a preferred embodiment, the skin contact layer is free of active agent, that is, is prepared without the addition of an active agent.

The skin contact layer may have an area weight of from 5 to 120 g/m². It is preferred, that the skin contact layer has an area weight of from 5 to 60 g/m², preferably of from 10 to 50 g/m², more preferably of from 20 to 40 g/m².

### Rivastigmine

The TTS according to the invention comprises a rivastigmine-containing layer structure, said rivastigmine containing layer structure comprising A) a backing layer; and B) a rivastigmine-containing layer comprising at least one acrylic polymer; and C) a skin contact layer comprising at least one styrene-isoprene-styrene block copolymer and at least one tackifier.

In one embodiment of the invention, the amount of rivastigmine contained in the rivastigmine-containing layer structure ranges from 0.5 to 5 mg/cm², preferably from 1 to 3 mg/cm².

In one embodiment of the invention, the rivastigmine-containing layer structure preferably contains a therapeutically effective amount of rivastigmine. More preferably, the therapeutically effective amount of rivastigmine is present in the rivastigmine-containing layer of the rivastigmine-containing layer structure. Preferably, the rivastigmine in the rivastigmine-containing layer structure is present in the form of the free base.

In one embodiment of the invention, at least 50 mol %, preferably at least 75 mol % of the total amount of rivastigmine in the TTS are present in the form of the free base. In a particular preferred embodiment, at least 90 mol %, preferably at least 95 mol %, more preferably at least 99 mol % of the total amount of rivastigmine in the TTS are present in the form of the free base. Thus, it is preferred that at least 50 mol %, preferably at least 75 mol % of the rivastigmine in the rivastigmine-containing layer are present in the form of the free base. In a particular preferred embodiment, at least 90 mol %, preferably at least 95 mol %, more preferably at least 99 mol % of the rivastigmine in the rivastigmine-containing layer are present in the form of the free base. In certain embodiments, the rivastigmine-containing layer is free of rivastigmine salts.

In certain embodiments, the amount of rivastigmine in the rivastigmine-containing layer ranges from 20 to 40% by weight, preferably from 25 to 35% by weight, most preferably rivastigmine is present in the rivastigmine-containing layer in an amount of 30% by weight, based on the total weight of the rivastigmine-containing layer.

In certain embodiments, the amount of rivastigmine contained in the rivastigmine-containing layer ranges from 1 to 72 mg, preferably from 2 to 36 mg, depending on the patch size. In a patch of the size of e.g. 5 cm², the amount of rivastigmine contained in the rivastigmine-containing layer ranges from 3 to 15 mg, preferably from 6 to 12 mg.

In one embodiment of the invention, the rivastigmine-containing layer is obtainable by dissolving or dispersing the rivastigmine in the form of the free base. If the rivastigmine-containing layer is a rivastigmine-containing matrix layer, said layer is preferably obtainable by dissolving or dispersing the rivastigmine in the form of the free base in the polymeric carrier, which particularly preferably comprises the at least one acrylic polymer.

In one embodiment, the rivastigmine-containing layer comprises a pharmaceutically acceptable salt of rivastigmine, such as rivastigmine hydrochloride or rivastigmine tartrate.

However, it is preferred according to the invention that the rivastigmine in the rivastigmine-containing layer is present in the form of the free base.

In certain embodiments, the rivastigmine has a purity of at least 95%, preferably of at least 98%, and more preferably of at least 99% as determined by quantitative titration according to Ph.Eur. 2.2.20 Assay in the Hyoscine Monography.

### Acrylic Polymers

According to the invention, the TTS comprises at least one acrylic polymer in the rivastigmine-containing layer.

In certain embodiments, the acrylic polymer is an acrylic pressure-sensitive adhesive.

Acrylic pressure-sensitive adhesives are usually supplied and used in solvents like n-heptane and ethyl acetate. The solids content of the pressure-sensitive adhesives is usually between 20% and 80%.

Acrylic pressure-sensitive adhesives may also be referred to as acrylate-based pressure-sensitive adhesives, or pressure-sensitive adhesives based on acrylates. Pressure-sensitive adhesives based on acrylates may have a solids content preferably between 20% and 60%. Such acrylate-based pressure-sensitive adhesives may or may not comprise functional groups such as hydroxy groups, carboxylic acid groups, neutralized carboxylic acid groups and mixtures thereof. Thus, the term “functional groups” in particular refers to hydroxy- and carboxylic acid groups, and deprotonated carboxylic acid groups.

Corresponding commercial products are available e.g. from Henkel under the tradename Duro Tak®. Such acrylate-based pressure-sensitive adhesives are based on monomers selected from one or more of acrylic acid, butylacrylate, 2-ethylhexylacrylate, glycidylmethacrylate, 2-hydroxyethylacrylate, methylacrylate, methylmethacrylate, t-octylacrylamide and vinylacetate, and are provided in ethyl acetate, heptanes, n-heptane, hexane, methanol, ethanol, isopropanol, 2,4-pentanedione, toluene or xylene or mixtures thereof.

Specific acrylate-based pressure-sensitive adhesives are available as:
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (a copolymer based on acrylic acid, 2-ethylhexylacrylate, glycidylmethacrylate and methylacrylate, provided as a solution in ethyl acetate and hexane),
Duro-Tak™ 87-4098 (a copolymer based on 2-ethylhexyl-acrylate and vinyl acetate, provided as a solution in ethyl acetate).

In a preferred embodiment of the invention, the acrylic polymer in the rivastigmine-containing layer is the acrylate-based pressure-sensitive adhesive Duro-Tak^{TM}384-2353, a copolymer based on acrylic acid, 2-ethylhexylacrylate, glycidylmethacrylate and methylacrylate, provided as a solution in ethyl acetate and hexane.

Additional polymers may also be added to enhance cohesion and/or adhesion.

### Styrene-Isoprene-Styrene Block Copolymers

According to the invention, the TTS comprises at least one styrene-isoprene-styrene block copolymer in the skin contact layer. In particular, the at least one styrene-isoprene-styrene block copolymer is a pressure-sensitive adhesives based on styrene-isoprene-styrene block copolymers.

Suitable styrene-isoprene-styrene copolymers according to the invention are commercially available e.g. under the brand names JSR-SIS.

In certain embodiments of the invention, the at least one styrene-isoprene-styrene block copolymer comprises styrene blocks and isoprene blocks in a ratio of from about 10:90 (w/w) to about 30:70 (w/w), preferably in a ratio of about 15:85 (w/w) or about 22:78 (w/w).

In certain embodiments of the invention, the at least one styrene-isoprene-styrene block copolymer is obtainable by polymerisation of three blocks of polystyrene, polyisoprene and polystyrene and has an average molecular weight of from about 100,000 to 200,000.

Specific styrene-isoprene-styrene block copolymer-based pressure-sensitive adhesives are available under the tradenames JSR-SIS5229 and JSR-SIS5002.

Additional polymers may also be added to enhance cohesion and/or adhesion.

### Tackifiers

According to the invention, the TTS comprises at least one tackifier in the skin contact layer. In certain embodiments of the invention, the at least one tackifier is an alicyclic saturated hydrocarbon resin, a hydrogenated rosin glycerol ester, paraffinum liquidum, or a mixture thereof. For example, the at least one tackifier may be a mixture comprising an alicyclic saturated hydrocarbon resin and paraffinum liquidum, or a mixture comprising a hydrogenated rosin glycerol ester and paraffinum liquidum.

Alicyclic saturated hydrocarbon resins are described in detail in the respective monograph in the Japanese pharmacopeia. In certain embodiments of the invention, the alicyclic saturated hydrocarbon resin is obtainable from polymerisation of an unsaturated hydrocarbon obtainable prepared by the decomposition of petroleum naphtha at elevated temperatures.

A specific alicyclic saturated hydrocarbon resin is available from Arakawa Europe under the tradename Arkon P-100 and has the chemical structure as detailed below.

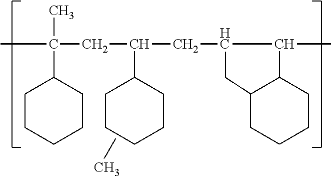

Hydrogenated rosin glycerol esters are described in detail in the respective monograph in the Japanese pharmacopeia. In certain embodiments of the invention, the hydrogenated rosin glycerol ester is a solid resin obtainable from the hydrogenation of rosin, followed by esterification with glycerin.

A specific hydrogenated rosin glycerol ester is available from Arakawa Europe under the tradename Pinecrystal KE-100 and has the chemical structure as detailed below.

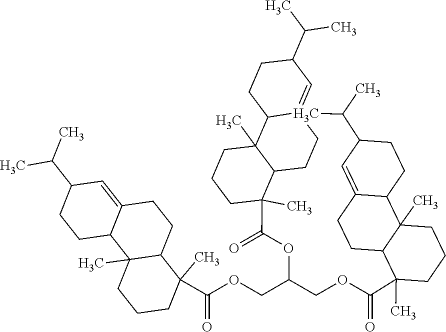

Paraffinum liquidum is a refined mixture of liquid, saturated hydrocarbons as defined in the European Pharmacopeia (Ph.Eur.).

According to certain embodiments of the invention, the at least one tackifier is contained in the skin contact layer in an amount from about 20 to about 80%, preferably from about 35 to about 65%.

### Further Additives

The TTS according to the invention, and in particular the rivastigmine-containing layer may further comprise at least one additive or excipient. Said additives or excipients are preferably selected from the group consisting of crystallization inhibitors, solubilizers, fillers, substances for skincare, pH regulators, preservatives, tackifiers, softeners, stabilizers, and permeation enhancers, in particular from crystallization inhibitors, substances for skincare, tackifiers, softeners, stabilizers, and permeation enhancers. More preferably, said additives are selected from the group consisting of crystallization inhibitors, solubilizers, fillers, substances for skincare, pH regulators, preservatives, tackifiers, softeners, stabilizers, and permeation enhancers, in particular from crystallization inhibitors, substances for skincare, tackifiers, softeners, and stabilizers. Such additives may be present in the rivastigmine-containing layer in an amount of from 0.001 to 15% by weight, e.g. from 1 to 10% by weight or from 0.01 to 5% by weight, based on the total weight of the rivastigmine-containing layer.

In certain preferred embodiments, the rivastigmine-containing layer does not comprise further additives, in particular the rivastigmine-containing layer does not comprise a permeation enhancer or solubilizer.

It should be noted that in pharmaceutical formulations, the formulation components are categorized according to their physicochemical and physiological properties, and in accordance with their function. This means in particular that a substance or a compound falling into one category is not excluded from falling into another category of formulation component. E.g. a certain polymer can be a crystallization inhibitor but also a tackifier. Some substances may e.g. be a typical softener but at the same time act as a permeation enhancer. The skilled person is able to determine based on his or her general knowledge in which category or categories of formulation component a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

In one embodiment, the rivastigmine-containing layer further comprises a crystallization inhibitor. In some embodiments, the crystallization inhibitor can be present in an amount of from 0.5 to 10% by weight based on the total weight of the rivastigmine-containing layer. Suitable examples of crystallization inhibitors include polyvinylpyrrolidone, vinyl acetate/vinylpyrrolidone copolymer and cellulose derivatives. The crystallization inhibitor is preferably polyvinylpyrrolidone, more preferably soluble polyvinylpyrrolidone. The crystallization inhibitor may increase the solubility of the active agent or inhibit the crystallization of the active agent.

In one embodiment, the rivastigmine-containing layer further comprises a stabilizer, wherein the stabilizer is preferably selected from tocopherol and ester derivatives thereof and ascorbic acid and ester derivatives thereof. In some embodiments, the stabilizer can be present in an amount of from 0.001 to 2.0%, preferably from 0.01 to 1.0% by weight based on the total weight of the rivastigmine-containing layer. In some embodiments, preferred stabilizers include sodium metabisulfite, ascorbyl esters of fatty acids such as ascorbyl palmitate, ascorbic acid, butylated hydroxytoluene, tocopherol, tocopheryl acetate and tocopheryl linoleate. Preferred stabilizers include ascorbyl esters of fatty acids, ascorbic acid, tocopherol, tocopheryl acetate and tocopheryl linoleate. Particularly preferred is tocopherol. Also particularly preferred is a combination of tocopherol and ascorbyl palmitate.

In one embodiment, the rivastigmine-containing layer further comprises a softener/plasticizer. Exemplary softeners/plasticizers include linear or branched, saturated or unsaturated alcohols having 6 to 20 carbon atoms, triglycerides and polyethylene glycols.

In one embodiment, the rivastigmine-containing layer further comprises a solubilizer. The solubilizer preferably improves the solubility of the rivastigmine in the rivastigmine-containing layer. Preferred solubilizers include, e.g., glycerol-, polyglycerol-, propylene glycol- and polyoxyethylene-esters of medium chain and/or long chain fatty acids, such as glyceryl mono lino leate, medium chain glycerides and medium chain triglycerides, non-ionic solubilizers made by reacting castor oil with ethylene oxide, and any mixtures thereof which may further contain fatty acids or fatty alcohols; cellulose and methylcellulose and derivatives thereof such as hydroxypropylcellulose and hypromellose acetate succinate; various cyclodextrins and derivatives thereof; non-ionic tri-block copolymers having a central hydrophobic chain of polyoxypropylene flanked by two hydrophilic chains of polyoxyethylene known as poloxamers;

water-soluble derivatives of vitamin E; pharmaceutical graded or agglomerated spherical isomalt; a polyethylene glycol, polyvinyl acetate and polyvinylcaprolactame-based graft copolymer, also abbreviated as PVAc-PVCap- PEG and known as Soluplus®; purified grades of naturally derived castor oil, of polyethylene glycol 400, of polyoxyethylene sorbitan monooleate (such as polysorbate 80) or of propylene glycols; diethylene glycol monoethyl ether; glucono-delta-lactone; maize and potato starch; as well as any of the below mentioned soluble polyvinylpyrrolidones, but also insoluble/cross-linked polyvinylpyrrolidones such as crospovidones.

However, also the permeation enhancers mentioned below can act as solubilizers.

In one embodiment, the rivastigmine-containing layer further comprises a pH regulator. Suitable pH regulators include mild acids and bases including amine derivatives, inorganic alkali derivatives, and polymers with basic or acidic functionality.

In one embodiment, the rivastigmine-containing layer further comprises a preservative. Suitable preservatives include parabens, formaldehyde releasers, isothiazolinones, phenoxyethanol, and organic acids such as benzoic acid, sorbic acid, levulinic acid and anisic acid.

In one embodiment, the rivastigmine-containing layer further comprises a substance for skincare. Such substances may be used to avoid or reduce skin irritation as detectable by the dermal response score. Suitable substances for skincare include sterol compounds such as cholesterol, dexpanthenol, alpha-bisabolol, and antihistamines. Substances for skincare are preferably used in amounts of from 1 to 10% by weight based on the total weight of the rivastigmine-containing layer.

The term “soluble polyvinylpyrrolidone” refers to polyvinylpyrrolidone, also known as povidone, which is soluble with more than 10% in at least ethanol, preferably also in water, diethylene glycol, methanol, n-propanol, 2 propanol, n-butanol, chloroform, methylene chloride, 2-pyrrolidone, macrogol 400, 1,2 propylene glycol, 1,4 butanediol, glycerol, triethanolamine, propionic acid and acetic acid. Examples of polyvinylpyrrolidones which are commercially available include Kollidon® 12 PF, Kollidon® 17 PF, Kollidon® 25, Kollidon® 30 and Kollidon0 90 F supplied by BASF, or povidone K9OF. The different grades of Kollidon® are defined in terms of the K-Value reflecting the average molecular weight of the polyvinylpyrrolidone grades. Kollidon® 12 PF is characterized by a K-Value range of 10.2 to 13.8, corresponding to a nominal K-Value of 12. Kollidon® 17 PF is characterized by a K-Value range of 15.3 to 18.4, corresponding to a nominal K-Value of 17. Kollidon® 25 is characterized by a K-Value range of 22.5 to 27.0, corresponding to a nominal K-Value of 25, Kollidon® 30 is characterized by a K-Value range of 27.0 to 32.4, corresponding to a nominal K-Value of 30. Kollidon® 90 F is characterized by a K-Value range of 81.0 to 97.2, corresponding to a nominal K-Value of 90. Preferred Kollidon® grades are Kollidon® 12 PF, Kollidon® 30 and Kollidon® 90 F.

Within the meaning of this invention, the term “K-Value” refers to a value calculated from the relative viscosity of polyvinylpyrrolidone in water according to the European Pharmacopoeia (Ph.Eur.) and USP monographs for “Povidone”.

Fillers such as silica gels, titanium dioxide and zinc oxide may be used in conjunction with the polymer in order to influence certain physical parameters, such as cohesion and bond strength, in the desired way.

In one embodiment, the rivastigmine-containing layer further comprises a permeation enhancer. Permeation enhancers are substances, which influence the barrier properties of the stratum corneum in the sense of increasing the active agent permeability. Some examples of permeation enhancers are polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin; polar solvents such as dimethyldecylphosphoxide, methylcetylsulfoxide, dimethylaurylamine, dodecyl pyrrolidone, isosorbitol, dimethylacetonide, dimethylsulfoxide, decylmethylsulfoxide, and dimethylformamide; salicylic acid; amino acids; benzyl nicotinate; and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate.

If the rivastigmine-containing layer further comprises a permeation enhancer, the permeation enhancer is preferably selected from diethylene glycol monoethyl ether (transcutol), diisopropyl adipate, isopropyl myristate, isopropyl palmitate, lauryl lactate, and dimethylpropylene urea.

It has been found that the TTS provides sufficient permeability of the active agent even if no permeation enhancer is present. Therefore, in certain embodiments of the invention, the rivastigmine-containing layer does not comprise a permeation enhancer or solubilizer.

### Release Characteristics

The TTS in accordance with the invention are designed for transdermally administering rivastigmine to the systemic circulation for a predefined extended period of time, preferably for 24 hours.

In one embodiment, the TTS according to the invention provides by transdermal delivery a mean release rate of from 150 to 3500 μg/cm²*day, preferably from 400 to 2000 μg/cm²*day rivastigmine over about 24 hours of administration.

In one embodiment, the TTS according to the invention provides by transdermal delivery from 2 to 20 mg of rivastigmine at an approximately constant rate, during an administration period of the TTS to the skin of the patient for about 24 hours.

In one embodiment, the transdermal therapeutic system according to the invention provides a cumulative permeated amount of rivastigmine as measured in a Franz diffusion cell with an EVA membrane of about 300 μg/cm² to 1200 μg/cm² over a time period of 24 hours.

In one embodiment, the transdermal therapeutic system according to the invention provides a permeated amount of rivastigmine as measured in a Franz diffusion cell with EVA-membrane (9% vinyl acetate Cotran 9702 von 3M) of 0μg/cm² to 240 μg/cm² in the first 3 hours, 80 μg/cm² to 350 μg/cm² from hour 3 to hour 8, 210 μg/cm² to 560 μg/cm² from hour 8 to hour 24.

### Method of Treatment/Medical Use

In accordance with a specific aspect of the present invention, the TTS according to the invention is for use in a method of treating a human patient, preferably for use in a method of preventing, treating, or delaying of progression of Alzheimer's disease, dementia associated with Parkinson's disease, and/or symptoms of traumatic brain injury. According to another specific aspect of the present invention, the TTS is for use in a method of treating a human patient, preferably for use in a method of treating mild to moderate dementia caused by Alzheimer's or Parkinson's disease.

In one embodiment, the TTS according to the invention is for use in a method of treating a human patient, preferably for use in a method of treating a human patient, preferably for use in a method of preventing, treating, or delaying of progression of Alzheimer's disease, dementia associated with Parkinson's disease, and/or symptoms of traumatic brain injury, or for use in a method of treating a human patient, preferably for use in a method of treating mild to moderate dementia caused by Alzheimer's or Parkinson's disease, wherein the transdermal therapeutic system is applied to the skin of the patient for a dosing interval of at least 24 hours, preferably about 24 hours.

In one embodiment, the TTS according to the invention is for use in a method of treating a human patient, preferably for use in a method of treating a human patient, preferably for use in a method of preventing, treating, or delaying of progression of Alzheimer's disease, dementia associated with Parkinson's disease, and/or symptoms of traumatic brain injury, or for use in a method of treating a human patient, preferably for use in a method of treating mild to moderate dementia caused by Alzheimer's or Parkinson's disease, wherein the transdermal therapeutic system is applied to the skin of the patient for a dosing interval of at least 72 hours, preferably about 84 hours.

In certain embodiments, the present invention relates to a method of treating a human patient, in particular preventing, treating, or delaying of progression of Alzheimer's disease, dementia associated with Parkinson's disease, and/or symptoms of traumatic brain injury, by applying a transdermal therapeutic system as defined within the invention to the skin of the patient. In another certain embodiment, the present invention relates to a method of treating a human patient, in particular treating a mild to moderate dementia caused by Alzheimer's and Parkinson's disease, by applying a transdermal therapeutic system as defined within the invention to the skin of the patient.

In one embodiment, the present invention relates to a method of treating a human patient, in particular preventing, treating, or delaying of progression of Alzheimer's disease, dementia associated with Parkinson's disease, and/or symptoms of traumatic brain injury, or a method of treating a human patient, in particular treating a mild to moderate dementia caused by Alzheimer's and Parkinson's disease, wherein the transdermal therapeutic system is applied to the skin of the patient for a dosing interval of at least 24 hours, preferably about 24 hours.

In one embodiment, the present invention relates to a method of treating a human patient, in particular preventing, treating, or delaying of progression of Alzheimer's disease, dementia associated with Parkinson's disease, and/or symptoms of traumatic brain injury, or a method of treating a human patient, in particular treating a mild to moderate dementia caused by Alzheimer's and Parkinson's disease, wherein the transdermal therapeutic system is applied to the skin of the patient for a dosing interval of at least 72 hours, preferably about 84 hours.

In connection with the above uses and methods of treatment, the TTS according to the invention is preferably applied to at least one body surface on the subject selected from the upper outer art, upper chest, upper back or the side of the chest for the defined dosing intervals.

The preferred application time of a TTS according to the invention is at least 24 hours, preferably about 24 hours (1 day) or about 84 hours (3.5 days), particularly preferably about 24 hours. After this time, the TTS may be removed, and optionally a new TTS may be applied, so as to allow an around-the-clock treatment.

### Process of Manufacture

The invention further relates to a process of manufacture of a rivastigmine-containing layer, preferably a rivastigmine-containing matrix layer, for use in a transdermal therapeutic system.

In accordance with the invention, the process for manufacturing a transdermal therapeutic system according to the invention comprises the steps of:
1) providing a rivastigmine-containing coating composition by combining at least the components
   i) rivastigmine; and
   ii) at least one acrylic polymer;
2) coating the rivastigmine-containing coating composition onto a film in an amount to provide the desired area weight,
3) drying the coated rivastigmine-containing coating composition to provide the rivastigmine-containing layer,
4) providing an additional coating composition for an additional skin contact layer by combining at least the components
   a) at least one styrene-isoprene-styrene block copolymer; and
   b) at least one tackifier;
5) coating and drying the additional coating composition according to steps 2 and 3, wherein the film is a release liner,
6) laminating the adhesive side of the skin contact layer onto the adhesive side of the rivastigmine-containing layer to provide a rivastigmine-containing layer structure with the desired area of release,
7) punching the individual systems from the rivastigmine-containing layer structure,
8) optionally adhering to the individual systems a rivastigmine-free self-adhesive layer structure comprising also a backing layer and a rivastigmine-free pressure-sensitive adhesive layer and which is larger than the individual systems of rivastigmine-containing self-adhesive layer structure.

In step 1) of the above process of manufacture, the rivastigmine is preferably dispersed to obtain a homogenous coating composition.

In certain embodiments of the present invention, the acrylic polymer is provided as a solution, wherein the solvent is ethyl acetate or n-heptane. Preferably ethyl acetate is used. Preferably, the acrylic polymer has a solids content of from 20 to 70% by weight.

In step 3) of the above process of manufacture, drying is performed preferably at a temperature of from 20 to 90° C., more preferably from 40 to 70° C.

In certain embodiments of the present invention, the film in step 2) is a release liner, the rivastigmine-containing layer is laminated after step 3) to a backing layer, and the release liner of step 2) is removed before step 6).

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Comparative Example 1

Comparative Example 1 (Comp. 1) is equivalent to the commercially available rivastigmine-containing TTS product Exelon®, having a rivastigmine-containing acrylic based layer (60 g/m²) and a rivastigmine-free silicone based skin contact layer (30 g/m²), but includes a transparent backing layer instead of the beige backing layer of the commercial product by Novartis.

The permeated amount of the commercially available Exelon® TTS as well as the stability in terms of the rivastigmine content, the adhesion force, the peel force and the in vitro release were determined in accordance with Examples 6 and 7, respectively.

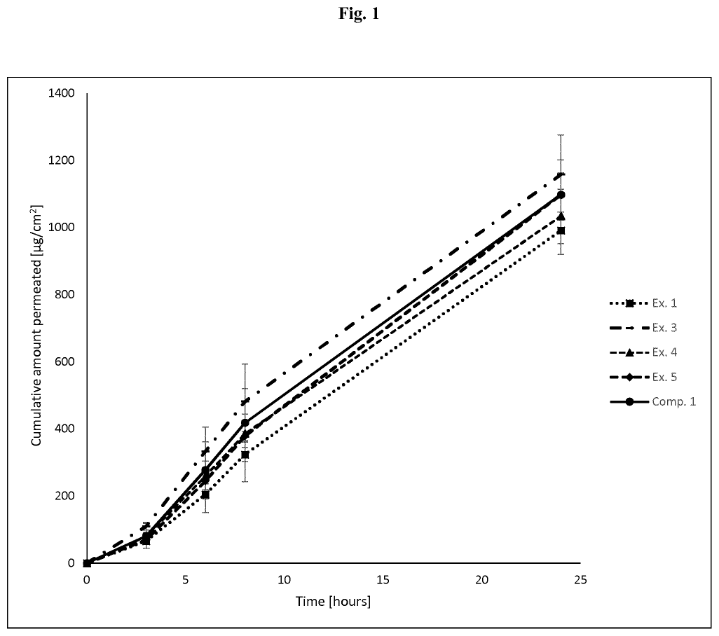

The results are shown in Tables 6 to 7.18 and in FIGS. 1 to 4.

### Example 1

### Coating Composition

The formulation of the rivastigmine-containing coating composition of Example 1 is summarized in Table 1.1. The %-values refer to the amounts in % by weight.

TABLE 1.1

| Ingredient (Trade Name) | Amt [kg] | Solids [%] |
|---|---|---|
| Rivastigmine base | 54.00 | 30.00 |
| Polybutylmethacrylate, methylmethacrylate | 36.00 | 20.00 |
| Acrylic adhesive in ethyl | 89.82 | 49.90 |
| acetate, solids content | (239.52 with | |
| of 37.5% (e.g. Durotak ® 387-2353) | ethyl acetate) | |
| Alpha-tocopherol | 0.18 | 0.10 |
| Total | 180.00 | 100.0 |

| | |
|---|---|
| Area Weight [g/m²] | 60 |

### Preparation of the Coating Composition

A beaker was loaded with the acrylic pressure-sensitive adhesive Durotak 387-2353. The rivastigmine base was added under stirring. The mixture was stirred at about 800 rpm until a homogenous mixture was obtained (at least 20 min).

### Coating of the Coating Composition

The resulting rivastigmine-containing coating composition was coated within less than 24 h after the rivastigmine-containing mixture was finished on an abhesively equipped foil (Scotchpak 9755 AB1F) using hand over knife lab coating equipment, using an erichson coater. The solvent was removed by drying in a first step at about room temperature (23 ±2° C.) for about 10 min, followed by a second drying step at about 60° C. for about 20 min.

The coating thickness was chosen such that removal of the solvent results in an area weight of the rivastigmine-containing layer of about 60.0 g/m². The dried film was then laminated with a backing layer (FO PET 23 gm transparent).

### Skin Contact Layer

The formulation of the skin contact layer of Example 1 is summarized in Table 1.2. The %-values refer to the amounts in % by weight.

TABLE 1.2

| Ingredient (Trade Name) | Amt [g] | Solids [%] |
|---|---|---|
| Styrene isoprene block copolymer in benzene | 64.94 | 49.95 |
| (JSR SIS 5229) | | |
| Alicyclic saturated hydrocarbon resin (Arkon | 64.94 | 49.95 |
| P-100) | | |
| Alpha-tocopherol | 0.13 | 0.10 |
| Total | 130.01 | 100.0 |

| | |
|---|---|
| Area Weight [g/m²] | 30 |

### Preparation of the Skin Contact Layer

A beaker was loaded with the styrene isoprene block copolymer and the alicyclic saturated hydrocarbon resin. The mixture was stirred at about 800 rpm until a homogenous mixture was obtained (at least 20 min).

The resulting composition was coated within less than 24 h after the mixture was finished on an abhesively equipped foil (Scotchpak 9755 AB1F) using hand over knife lab coating equipment, using an erichson coater. The solvent was removed by drying in a first step at about room temperature (23±2° C.) for about 10 min, followed by a second drying step at about 60° C. for about 20 min.

The coating thickness was chosen such that removal of the solvent results in an area weight of the skin contact layer of about 30.0 g/m². The abhesively equipped foil of the rivastigmine-containing layer was removed and the dried skin contact layer was then laminated on top of it to form a rivastigmine-containing self-adhesive layer structure.

### Preparation of the TTS

The individual systems (TTS) were then punched out from the rivastigmine-containing self-adhesive layer structure and sealed into pouches of the primary packaging material.

### Example 2

### Rivastigmine-Containing Layer

The formulation of the rivastigmine-containing layer of Example 2 corresponds to the rivastigmine-containing layer of Example 1 as summarized in Table 1.1.

### Skin Contact Layer

The formulation of the skin contact layer of Example 2 is summarized in Table 2. The %-values refer to the amounts in % by weight.

TABLE 2

| Ingredient (Trade Name) | Amt [g] | Solids [%] |
|---|---|---|
| Styrene isoprene block copolymer in benzene | 54.15 | 41.7 |
| (JSR SIS 5229) | | |
| Alicyclic saturated hydrocarbon resin (Arkon | 66.11 | 50.9 |
| P-100) | | |
| Paraffinum liquidum | 9.62 | 7.4 |
| Alpha-tocopherol | 0.13 | 0.10 |
| Total | 130.01 | 100.0 |

| | |
|---|---|
| Area Weight [g/m²] | 30 |

### Preparation of the Skin Contact Layer

A beaker was loaded with the styrene isoprene block copolymer, the alicyclic saturated hydrocarbon resin and paraffinum liquidum. The mixture was stirred at about 800 rpm until a homogenous mixture was obtained (at least 20 min).

The resulting composition was coated within less than 24 h after the mixture was finished on an abhesively equipped foil (Scotchpak 9755 AB1F) using hand over knife lab coating equipment, using an erichson coater. The solvent was removed by drying in a first step at about room temperature (23±2° C.) for about 10 min, followed by a second drying step at about 60° C. for about 20 min.

The coating thickness was chosen such that removal of the solution results in an area weight of the skin contact layer of about 30.0 g/m². The abhesively equipped foil of the rivastigmine-containing layer was removed and the dried skin contact layer was then laminated on top of it to form a rivastigmine-containing self-adhesive layer structure.

### Preparation of the TTS

The individual systems (TTS) were then punched out from the rivastigmine-containing self-adhesive layer structure and sealed into pouches of the primary packaging material.

### Example 3

### Rivastigmine-Containing Layer

The formulation of the rivastigmine-containing layer of Example 3 corresponds to the rivastigmine-containing layer of Example 1 as summarized in Table 1.1.

### Skin Contact Layer

The formulation of the skin contact layer of Example 3 is summarized in Table 3. The %-values refer to the amounts in % by weight.

TABLE 3

| Ingredient (Trade Name) | Amt [g] | Solids [%] |
|---|---|---|
| Styrene isoprene block copolymer in benzene | 64.94 | 49.95 |
| (JSR SIS 5229) | | |
| Hydrogenated rosin glycerol ester (Pinecrystal | 64.94 | 49.95 |
| KE-311) | | |
| Alpha-tocopherol | 0.13 | 0.10 |
| Total | 130.01 | 100.0 |

| | |
|---|---|
| Area Weight [g/m²] | 30 |

### Preparation of the Skin Contact Layer

A beaker was loaded with the styrene isoprene block copolymer, the hydrogenated rosin glycerol ester and alpha-tocopherol. The mixture was stirred at about 800 rpm until a homogenous mixture was obtained (at least 20 min).

The resulting composition was coated within less than 24 h after the mixture was finished on an abhesively equipped foil (Scotchpak 9755 AB1F) using hand over knife lab coating equipment, using an erichson coater. The solvent was removed by drying in a first step at about room temperature (23 ±2° C.) for about 10 min, followed by a second drying step at about 60° C. for about 20 min.

The coating thickness was chosen such that removal of the solution results in an area weight of the skin contact layer of about 30.0 g/m². The abhesively equipped foil of the rivastigmine-containing layer was removed and the dried skin contact layer was then laminated on top of it to form a rivastigmine-containing self-adhesive layer structure.

### Preparation of the TTS

The individual systems (TTS) were then punched out from the rivastigmine-containing self-adhesive layer structure and sealed into pouches of the primary packaging material.

### Example 4

### Rivastigmine-Containing Layer

The formulation of the rivastigmine-containing layer of Example 4 corresponds to the rivastigmine-containing layer of Example 1 as summarized in Table 1.1.

### Skin Contact Layer

The formulation of the skin contact layer of Example 4 is summarized in Table 4. The %-values refer to the amounts in % by weight.

TABLE 4

| Ingredient (Trade Name) | Amt [g] | Solids [%] |
|---|---|---|
| Styrene isoprene block copolymer in benzene | 64.87 | 49.90 |
| (JSR SIS 5229) | | |
| Alicyclic saturated hydrocarbon resin (Arkon | 32.50 | 25.00 |
| P-100) | | |
| Hydrogenated rosin glycerol ester (Pinecrystal | 32.50 | 25.00 |
| KE-311) | | |
| Alpha-tocopherol | 0.13 | 0.10 |
| Total | 130.00 | 100.0 |

| | |
|---|---|
| Area Weight [g/m²] | 30 |

### Preparation of the Skin Contact Layer

A beaker was loaded with the styrene isoprene block copolymer, the alicyclic saturated hydrocarbon resin, the hydrogenated rosin glycerol ester and alpha-tocopherol. The mixture was stirred at about 800 rpm until a homogenous mixture was obtained (at least 20 min).

The resulting composition was coated within less than 24 h after the mixture was finished on an abhesively equipped foil (Scotchpak 9755 AB1F) using hand over knife lab coating equipment, using an erichson coater. The solvent was removed by drying in a first step at about room temperature (23 ±2° C.) for about 10 min, followed by a second drying step at about 60° C. for about 20 min.

The coating thickness was chosen such that removal of the solution results in an area weight of the skin contact layer of about 30.0 g/m². The abhesively equipped foil of the rivastigmine-containing layer was removed and the dried skin contact layer was then laminated on top of it to form a rivastigmine-containing self-adhesive layer structure.

### Preparation of the TTS

The individual systems (TTS) were then punched out from the rivastigmine-containing self-adhesive layer structure and sealed into pouches of the primary packaging material.

### Example 5

### Rivastigmine-Containing Layer

The formulation of the rivastigmine-containing layer of Example 5 corresponds to the rivastigmine-containing layer of Example 1 as summarized in Table 1.1.

### Skin Contact Layer

The formulation of the skin contact layer of Example 5 is summarized in Table 5. The %-values refer to the amounts in % by weight.

TABLE 5

| Ingredient (Trade Name) | Amt [g] | Solids [%] |
|---|---|---|
| Styrene isoprene block copolymer in benzene | 54.15 | 41.65 |
| (JSR SIS 5229) | | |
| Hydrogenated rosin glycerol ester (Pinecrystal | 66.11 | 50.85 |
| KE-311) | | |
| Paraffinum liquidum | 9.62 | 7.40 |
| Alpha-tocopherol | 0.13 | 0.10 |
| Total | 130.01 | 100.0 |

| | |
|---|---|
| Area Weight [g/m²] | 30 |

### Preparation of the Skin Contact Layer

A beaker was loaded with the styrene isoprene block copolymer, the hydrogenated rosin glycerol ester, the paraffinum liquidum and alpha-tocopherol. The mixture was stirred at about 800 rpm until a homogenous mixture was obtained (at least 20 min).

The resulting composition was coated within less than 24 h after the mixture was finished on an abhesively equipped foil (Scotchpak 9755 AB1F) using hand over knife lab coating equipment, using an erichson coater. The solvent was removed by drying in a first step at about room temperature (23 ±2° C.) for about 10 min, followed by a second drying step at about 60° C. for about 20 min.

The coating thickness was chosen such that removal of the solution results in an area weight of the skin contact layer of about 30.0 g/m². The abhesively equipped foil of the rivastigmine-containing layer was removed and the dried skin contact layer was then laminated on top of it to form a rivastigmine-containing self-adhesive layer structure.

### Preparation of the TTS

The individual systems (TTS) were then punched out from the rivastigmine-containing self-adhesive layer structure and sealed into pouches of the primary packaging material.

### Example 6

### Measurement of Permeated Amount

The permeated amount of the TTS prepared according to Comparative Example 1 and Examples 1 and 3 to 5 was determined by experiments in accordance with the EMA Guideline on quality of transdermal patches (adopted October 23, 2014) carried out with a 10.0 ml Franz diffusion cell, wherein an EVA-membrane (9% vinyl acetate; Scotchpak Cotran 9702 from 3M) having a thickness of 50 gm was used. Diecuts with an area of release of 1.156 cm² were punched from the TTS. The permeated amount of rivastigmine in the receptor medium of the Franz diffusion cell (phosphate buffer solution pH 5.5 with 0.1% sodium azide as antibacteriological agent) at a temperature of 32±1° C. was measured.

The results are shown in Table 6 and FIG. 1.

TABLE 6
Cumulative amount permeated with SD [μg/cm²]

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 3 | 66 | 4 | n.d. | n.d. | 110 | 11 |
| 6 | 205 | 13 | n.d. | n.d. | 333 | 29 |
| 8 | 324 | 21 | n.d. | n.d. | 482 | 38 |
| 24 | 991 | 39 | n.d. | n.d. | 1158 | 44 |

Cumulative amount permeated with SD [μg/cm²]

| Elapsed | Ex. 4 (n = 3) | | Ex. 5 (n = 3) | | Comp. Ex. 1 (n = 3) | |
|---|---|---|---|---|---|---|
| time [h] | Amount | SD | Amount | SD | Amount | SD |
| 3 | 80 | 11 | 69 | 1 | 81 | 37 |
| 6 | 259 | 21 | 245 | 9 | 278 | 127 |
| 8 | 386 | 25 | 379 | 13 | 418 | 175 |
| 24 | 1034 | 12 | 1098 | 65 | 1098 | 178 |

### Example 7

### Stability

The stability of the TTS prepared according to Comparative Example 1 and Examples 1 to 5 was determined with regard to different parameters, namely the adhesion force, the peel force, the rivastigmine content and the in vitro release.

The respective measurements were performed after preparation of the TTS (initial).

Subsequently, the TTS were stored at different storage conditions and the respective measurements were repeated after 3 months and after 12 months.

The storage conditions were either 25° C. and 60% relative humidity (25° C./60% RH), or 30° C. and 75% relative humidity (30° C./75% RH), or 40° C. and 75% relative humidity (40° C./75% RH).

Measurements were repeated after 3 months (25° C./60% RH and 40° C./75% RH) and after 12 months (25° C./60% RH, 30° C./75% RH and 40° C./75% RH). For Examples 1 and 3, no measurements after 12 months were performed.

### Example 7A

### Measurement of the Adhesion Force and the Peel Force

The adhesion force and the peel force of the TTS prepared according to Comparative Example 1 and Examples 1 to 5 were determined.

Adhesion force tests were performed with the TTS using a tensile strength testing machine. Prior testing the samples were equilibrated 24 hours under controlled conditions at approx. room temperature (23±2° C.) and approx. 50% rh (relative humidity). The first millimeters of the abhesively equipped foil was pulled off and a splicing tape is applied to the opened adhesive side. Then, the abhesively foil was totally removed and the TTS was placed with the adhesive surface in longitudinal direction onto the center of the cleaned testing plate (aluminum or stainless steel). The testing plate was fixed to the lower clamp of the tensile strength machine. The machine was adjusted to zero, the splicing tape was gripped into the upper clamp of the machine. The pull angle was set to 90° . After measurement of the adhesion force of three samples, the mean value of the adhesion force was calculated. The measurement value is based on units “N/TTS” [N/TTS].

Peel force test were performed with the TTS using a tensile strength testing machine. Prior testing the samples were equilibrated 24 hours under controlled conditions at approx. room temperature (23±2° C.) and approx. 50% rh (relative humidity). Further, the samples were cut into pieces with a fixed width of 25 mm and a suitable length. The first millimeters of the abhesively equipped foil was pulled off and a splicing tape is applied to the opened adhesive side. Then, the abhesively foil was totally removed and the sample was placed with the adhesive surface in longitudinal direction onto the center of the cleaned testing plate (aluminum or stainless steel). The testing plate was fixed to the lower clamp of the tensile strength machine. The machine was adjusted to zero, the splicing tape was gripped into the upper clamp of the machine. The pull angle was set to 90° and the peel off velocity was 150 mm/min. After measurement of the peel force of three samples, the mean value of the peel force was calculated. The measurement value is based on units “cN/TTS” [cN/TTS].

The TTS were stored at different storage conditions as detailed above and measurements were repeated after 3 months and after 12 months.

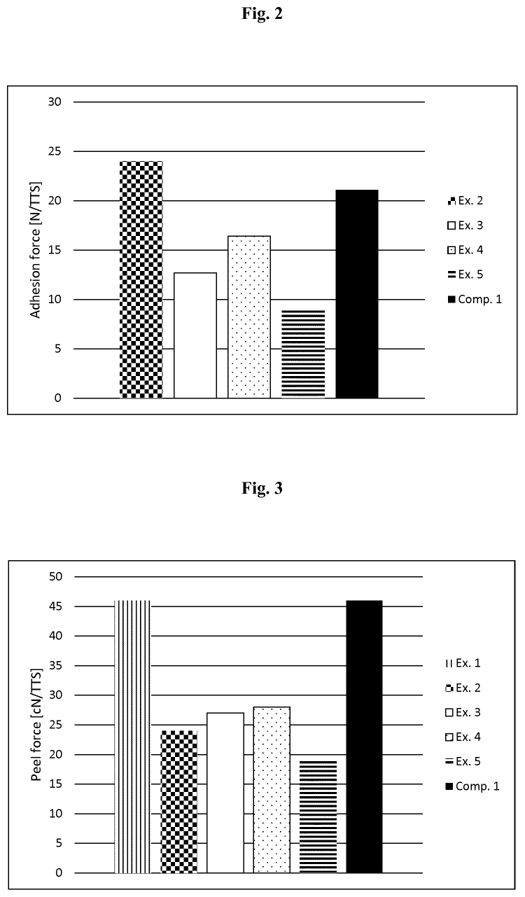

The results are shown in Tables 7.1 to 7.6. The intial adhesion force and the initial peel force after production, respectively, of Comparative Example 1 and Examples 1 to 5 are shown in FIGS. 2 and 3.

TABLE 7.1

| | | | | 30° C./ | | |
|---|---|---|---|---|---|---|
| | | 25° C./60% RH | | 75% RH | 40° C./75% RH | |
| | | 3 | 12 | 12 | 3 | 12 |
| Ex. 1 | initial | months | months | months | months | months |
| Adhesion | Test | Test | n.d. | n.d. | Test | n.d. |
| force | method | method | | | method | |
| [N/TTS] | not | not | | | not | |
| | suitable | suitable | | | suitable | |
| Peel | 46 | 48 | n.d. | n.d. | 47 | n.d. |
| force | | | | | | |
| [cN/TTS] | | | | | | |

TABLE 7.2

| | | 25° C./60% RH | | 30° C./75% RH | 40° C./75% RH | |
|---|---|---|---|---|---|---|
| | | 3 | 12 | 12 | 3 | 12 |
| Ex. 2 | initial | months | months | months | months | months |
| Adhesion | 24 | 21.9 | 24 | 25.5 | Partial | Partial |
| force | | | | | adhesive | adhesive |
| [N/TTS] | | | | | failure | failure |
| Peel | 24 | 30 | 35 | 32 | 29 | 29 |
| force | | | | | | |
| [cN/TTS] | | | | | | |

TABLE 7.3

| | | 25° C./60% RH | | 30° C./75% RH | 40° C./75% RH | |
|---|---|---|---|---|---|---|
| | | 3 | 12 | 12 | 3 | 12 |
| Ex. 3 | initial | months | months | months | months | months |
| Adhesion | 12.7 | 13.2 | n.d. | n.d. | 17.2 | n.d. |
| force | | | | | | |
| [N/TTS] | | | | | | |
| Peel | 27 | 30 | n.d. | n.d. | 26 | n.d. |
| force | | | | | | |
| [cN/TTS] | | | | | | |

TABLE 7.4

| | | 25° C./60% RH | | 30° C./ | 40° C./75% RH | |
|---|---|---|---|---|---|---|
| | | 3 | 12 | 75% RH | 3 | 12 |
| Ex. 4 | initial | months | months | 12 months | months | months |
| Adhesion | 16.4 | 18.3 | 17.3 | Partial | Partial | Partial |
| force | | | | adhesive | adhesive | adhesive |
| [N/TTS] | | | | failure | failure | failure |
| Peel | 28 | 30 | 34 | 34 | 33 | 31 |
| force | | | | | | |
| [cN/TTS] | | | | | | |

TABLE 7.5

| | | 25° C./60% RH | | 30° C./ | | |
|---|---|---|---|---|---|---|
| | | 3 | 12 | 75% RH | 40° C./75% RH | |
| Ex. 5 | initial | months | months | 12 months | 3 months | 12 months |
| Adhesion | 8.9 | 9 | 9.2 | 9.1 | 8.9 | 10.6 |
| force | | | | | | |
| [N/TTS] | | | | | | |
| Peel | 19 | 23 | 29 | 28 | 23 | 24 |
| force | | | | | | |
| [cN/TTS] | | | | | | |

TABLE 7.6

| | | | | 30° C./ | 40° C./75% RH | |
|---|---|---|---|---|---|---|
| Comp. | | 25° C./60% RH | | 75% RH | 3 | 12 |
| Ex. 1 | initial | 3 months | 12 months | 12 months | months | months |
| Adhesion | 21.1 | 16.8 | 14.8 | 11.7 | 16.5 | 9.2 |
| force | | | | | | |
| [N/TTS] | | | | | | |
| Peel force | 46 | 98 | 139 | 212 | 157 | 435 |
| [cN/TTS] | | | | | | |

### Example 7B

### Measurement of the Rivastigmine Content

The rivastigmine content of the TTS prepared according to Comparative Example 1 and

Examples 1 to 5 was determined by using a validated HPLC method (column: stainless steel column 150 mm×4.6 mm internal diameter, 5 μm particle size, C8 phase, e.g. YMC basic (Fa. YMC); column temperature: 30° C.; mobile phase: Acetonitrile/0.1 M KH₂PO₄/TEA 18:82:0.1 (v/v/v); flow rate: 1.2 ml/min; injection volume: 10 μL; Detection: UV at 264 nm, stop time: 8 min).

The results are shown in Tables 7.7 to 7.12.

TABLE 7.7

| | | 25° C./60% RH | | 30° C./ | 40° C./75% RH | |
|---|---|---|---|---|---|---|
| | | 3 | 12 | 75% RH | 3 | 12 |
| Ex. 1 | initial | months | months | 12 months | months | months |
| Rivastigmine | 66.7 ± | 97.2 ± 1.5 | n.d. | n.d. | 94.1 ± | n.d. |
| content [% | 1.5 | | | | 5.9 | |
| of label | | | | | | |
| claim] | | | | | | |

TABLE 7.8

| | | 25° C./60% RH | | 30° C./ | 40° C./75% RH | |
|---|---|---|---|---|---|---|
| | | 3 | 12 | 75% RH | 3 | 12 |
| Ex. 2 | initial | months | months | 12 months | months | months |
| Rivastigmine | 98.8 ± | 97.3 ± | 98.2 ± | 100.3 ± 1.7 | 92.9 ± | 100.4 ± 1.2 |
| content [% | 0.6 | 1.8 | 5.6 | | 6.9 | |
| of label | | | | | | |
| claim] | | | | | | |

TABLE 7.9

| | | 25° C./60% RH | | 30° C./ | 40° C./75% RH | |
|---|---|---|---|---|---|---|
| | | 3 | 12 | 75% RH | 3 | 12 |
| Ex. 3 | initial | months | months | 12 months | months | months |
| Rivastigmine | 99.8 ± | 97.1 ± | n.d. | n.d. | 96.0 ± | n.d. |
| content [% | 1.2 | 3.0 | | | 1.8 | |
| of label | | | | | | |
| claim] | | | | | | |

TABLE 7.10

| | | 25° C./60% RH | | 30° C./ | 40° C./75% RH | |
|---|---|---|---|---|---|---|
| | | 3 | 12 | 75% RH | 3 | 12 |
| Ex. 4 | initial | months | months | 12 months | months | months |
| Rivastigmine | 98.5 ± | 94.2 ± | 101.3 ± | 98.1 ± 1.6 | 95.9 ± | 96.2 ± 1.6 |
| content [% | 1.1 | 4.3 | 3.3 | | 1.0 | |
| of label | | | | | | |
| claim] | | | | | | |

TABLE 7.11

| | | 25° C./60% RH | | 30° C./ | 40° C./75% RH | |
|---|---|---|---|---|---|---|
| | | 3 | 12 | 75% RH | 3 | 12 |
| Ex. 5 | initial | months | months | 12 months | months | months |
| Rivastigmine | 98.9 ± | 95.0 ± | 98.5 ± | 98.9 ± 0.5 | 96.2 ± | 97.8 ± |
| content [% | 1.1 | 2.1 | 2.4 | | 1.6 | 1.3 |
| of label | | | | | | |
| claim] | | | | | | |

TABLE 7.12

| | | 25° C./60% RH | | 30° C./ | 40° C./75% RH | |
|---|---|---|---|---|---|---|
| | | 3 | 12 | 75% RH | 3 | 12 |
| Comp. Ex. 1 | initial | months | months | 12 months | months | months |
| Rivastigmine | 100.2 ± | 95.8 ± | 99.2 ± | 95.7 ± 2.5 | 91.5 ± | 94.0 ± |
| content [% | 2.4 | 2.6 | 2.2 | | 2.6 | 1.3 |
| of label | | | | | | |
| claim] | | | | | | |

### Example 7C

Measurement of the Rivastigmine in vitro Release Rate

The rivastigmine in vitro release rate from the TTS prepared according to Comparative Example 1 and Examples 1 to 5 was determined by experiments using the rotating cylinder apparatus of the Ph Eur/USP. The back of the TTS is affixed to the cylinder element using double sided adhesive tape. Following removal of the release liner, the cylinder is lowered into the dissolution medium (500 ml, degassed 0.9% sodium chloride solution at 32° C.) and rotated at 50 rpm. At 0.5, 2, 4, 7 and 24 hours, 4 ml samples are removed and analyzed using a validated HPLC method (column: Stainless steel column, 150mm×3.9mm I.D. packed with C18-Phase (e.g. Novapak C18, 4μm particle size, Waters) or equivalent column, column temperature: 20-25° C.; mobile phase: Acetonitrile/Water 20:80 (v/v) +0.35mL TEA per 100mL, pH 3.5; adjust the pH with phosphoric acid (85%), if necessary; flow rate: 1.0 ml/min; pressure: approx. 100 bar; injection volume: 20 μL; Detection: UV, 210 nm, stop time: 6 min).

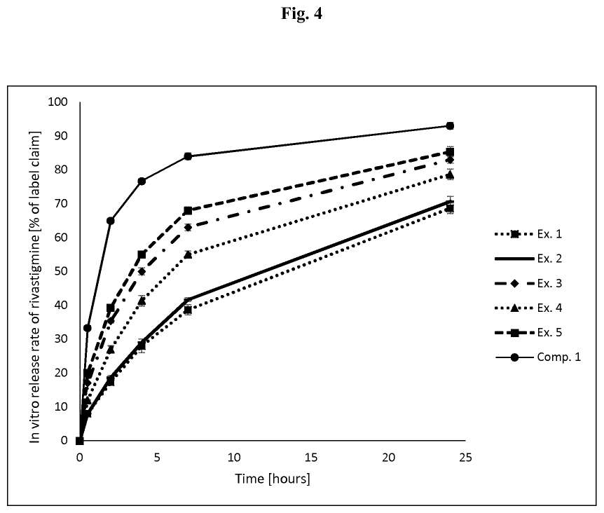

The results are shown in Tables 7.13 to 7.18. The initial (after production) rivastigmine in vitro release rate over 24 hours from Comparative Example 1 and Examples 1 to 5 is shown in FIG. 4.

TABLE 7.13
Rivastigmine in vitro release rate with SD [% of label claim]-initial

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 0.5 | 8.0 | 0.0 | 8.0 | 0.0 | 17.0 | 0.0 |
| 2 | 17.3 | 0.6 | 18.7 | 0.6 | 35.3 | 0.6 |
| 4 | 28.0 | 2.0 | 29.0 | 1.0 | 50.0 | 1.0 |
| 7 | 38.7 | 1.5 | 41.7 | 0.6 | 63.0 | 1.0 |
| 24 | 68.7 | 1.5 | 70.7 | 1.5 | 83.0 | 1.0 |
| Elapsed | Ex. 4 (n = 3) | | Ex. 5 (n = 3) | | Comp. Ex. 1 (n = 3) | |
| time [h] | Amount | SD | Amount | SD | Amount | SD |
| 0.5 | 12.0 | 0.0 | 20.0 | 0.0 | 33.3 | 0.6 |
| 2 | 27.0 | 1.0 | 39.3 | 0.6 | 65.0 | 0.0 |
| 4 | 41.3 | 1.5 | 55.0 | 1.0 | 76.7 | 0.6 |
| 7 | 55.0 | 1.0 | 68.0 | 1.0 | 84.0 | 1.0 |
| 24 | 78.7 | 1.5 | 85.3 | 1.5 | 93.0 | 1.0 |

TABLE 7.14
Rivastigmine in vitro release rate with SD [% of label claim]-
after storage for 3 months at 25° C./60% RH

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 0.5 | 7.7 | 0.6 | 8.0 | 0.0 | 18.7 | 0.6 |
| 2 | 17.0 | 1.0 | 18.3 | 0.6 | 36.0 | 0.0 |
| 4 | 26.7 | 1.5 | 28.3 | 0.6 | 49.7 | 0.6 |
| 7 | 37.3 | 2.1 | 39.7 | 1.5 | 61.7 | 0.6 |
| 24 | 55.3 | 15.9 | 67.7 | 1.5 | 78.7 | 0.6 |
| Elapsed | Ex. 4 (n = 3) | | Ex. 5 (n = 3) | | Comp. Ex. 1 (n = 3) | |
| time [h] | Amount | SD | Amount | SD | Amount | SD |
| 0.5 | 12.0 | 0.0 | 19.7 | 0.6 | 31.3 | 2.1 |
| 2 | 27.3 | 0.6 | 38.7 | 0.6 | 58.7 | 4.2 |
| 4 | 41.0 | 1.0 | 53.0 | 1.0 | 70.3 | 3.1 |
| 7 | 54.3 | 0.6 | 64.7 | 1.5 | 77.7 | 2.5 |
| 24 | 76.3 | 0.6 | 80.7 | 1.5 | 87.7 | 1.5 |

TABLE 7.15
Rivastigmine in vitro release rate with SD [% of label claim]-
after storage for 12 months at 25° C./60% RH

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 0.5 | n.d. | n.d. | 8.0 | 0.0 | n.d. | n.d. |
| 2 | n.d. | n.d. | 17.7 | 0.6 | n.d. | n.d. |
| 4 | n.d. | n.d. | 27.7 | 0.6 | n.d. | n.d. |
| 7 | n.d. | n.d. | 38.7 | 0.6 | n.d. | n.d. |
| 24 | n.d. | n.d. | 65.7 | 0.6 | n.d. | n.d. |
| Elapsed | Ex. 4 (n = 3) | | Ex. 5 (n = 3) | | Comp. Ex. 1 (n = 3) | |
| time [h] | Amount | SD | Amount | SD | Amount | SD |
| 0.5 | 11.3 | 0.6 | 17.3 | 0.6 | 29.0 | 3.6 |
| 2 | 25.3 | 0.6 | 33.0 | 1.0 | 53.7 | 6.1 |
| 4 | 38.0 | 1.0 | 46.3 | 1.5 | 64.0 | 5.6 |
| 7 | 50.3 | 1.5 | 57.7 | 1.5 | 72.0 | 4.6 |
| 24 | 73.0 | 1.0 | 75.7 | 1.2 | 82.3 | 3.5 |

TABLE 7.16
Rivastigmine in vitro release rate with SD [% of label claim]-
after storage for 12 months at 30° C./75% RH

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 0.5 | n.d. | n.d. | 8.3 | 0.6 | n.d. | n.d. |
| 2 | n.d. | n.d. | 17.7 | 0.0 | n.d. | n.d. |
| 4 | n.d. | n.d. | 27.0 | 1.0 | n.d. | n.d. |
| 7 | n.d. | n.d. | 38.0 | 1.0 | n.d. | n.d. |
| 24 | n.d. | n.d. | 64.0 | 1.0 | n.d. | n.d. |
| Elapsed | Ex. 4 (n = 3) | | Ex. 5 (n = 3) | | Comp. Ex. 1 (n = 3) | |
| time [h] | Amount | SD | Amount | SD | Amount | SD |
| 0.5 | 11.0 | 0.0 | 14.7 | 0.6 | 27.3 | 2.1 |
| 2 | 24.3 | 0.6 | 24.0 | 3.0 | 50.7 | 4.2 |
| 4 | 36.0 | 0.0 | 33.3 | 5.0 | 61.7 | 3.5 |
| 7 | 47.7 | 0.6 | 43.0 | 6.6 | 68.7 | 2.5 |
| 24 | 70.0 | 0.0 | 64.3 | 5.7 | 80.0 | 1.0 |

TABLE 7.17
Rivastigmine in vitro release rate with SD [% of label claim]-
after storage for 3 months at 40° C./75% RH

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 0.5 | 8.0 | 0.0 | 8.3 | 0.6 | 15.3 | 0.6 |
| 2 | 17.0 | 0.0 | 18.0 | 0.0 | 25.0 | 1.0 |
| 4 | 25.3 | 0.6 | 27.3 | 0.6 | 34.0 | 2.0 |
| 7 | 35.0 | 1.0 | 38.3 | 0.6 | 43.7 | 2.1 |
| 24 | 61.7 | 1.5 | 65.0 | 1.0 | 66.0 | 2.0 |
| Elapsed | Ex. 4 (n = 3) | | Ex. 5 (n = 3) | | Comp. Ex. 1 (n = 3) | |
| time [h] | Amount | SD | Amount | SD | Amount | SD |
| 0.5 | 11.7 | 0.6 | 14.3 | 0.6 | 30.3 | 0.6 |
| 2 | 22.3 | 2.5 | 21.3 | 1.5 | 56.3 | 0.6 |
| 4 | 31.0 | 5.0 | 28.0 | 2.0 | 67.0 | 1.0 |
| 7 | 40.7 | 7.0 | 36.0 | 3.0 | 73.0 | 1.0 |
| 24 | 63.3 | 7.5 | 58.0 | 4.0 | 82.7 | 1.5 |

TABLE 7.18
Rivastigmine in vitro release rate with SD [% of label claim]-
after storage for 12 months at 40° C./75% RH

| Elapsed | Ex. 1 (n = 3) | | Ex. 2 (n = 3) | | Ex. 3 (n = 3) | |
|---|---|---|---|---|---|---|
| time [h] | Amount | SD | Amount | SD | Amount | SD |
| 0.5 | n.d. | n.d. | 8.7 | 0.6 | n.d. | n.d. |
| 2 | n.d. | n.d. | 17.7 | 0.6 | n.d. | n.d. |
| 4 | n.d. | n.d. | 26.0 | 1.0 | n.d. | n.d. |
| 7 | n.d. | n.d. | 35.3 | 1.2 | n.d. | n.d. |
| 24 | n.d. | n.d. | 59.7 | 1.5 | n.d. | n.d. |
| Elapsed | Ex. 4 (n = 3) | | Ex. 5 (n = 3) | | Comp. Ex. 1 (n = 3) | |
| time [h] | Amount | SD | Amount | SD | Amount | SD |
| 0.5 | 11.3 | 0.6 | 13.3 | 0.6 | 23.7 | 0.6 |
| 2 | 20.0 | 2.0 | 19.0 | 1.0 | 45.0 | 1.0 |
| 4 | 26.0 | 3.0 | 23.7 | 1.5 | 56.0 | 1.0 |
| 7 | 33.0 | 5.0 | 29.7 | 2.5 | 63.0 | 1.0 |
| 24 | 52.0 | 7.5 | 47.3 | 5.0 | 75.7 | 0.6 |

### The Invention Relates in Particular to the Following Further Items:

1. Transdermal therapeutic system for the transdermal administration of rivastigmine comprising a rivastigmine-containing layer structure, said rivastigmine-containing layer structure comprising:

A) a backing layer;

B) a rivastigmine-containing layer comprising at least one acrylic polymer; and

C) a skin contact layer comprising at least one styrene-isoprene-styrene block copolymer and at least one tackifier.
2. Transdermal therapeutic system according to item 1, wherein the rivastigmine-containing layer is a rivastigmine-containing matrix layer comprising:

i) rivastigmine; and

ii) the acrylic polymer.
3. Transdermal therapeutic system according to item 1 or 2, wherein the rivastigmine-containing layer structure contains a therapeutically effective amount of rivastigmine.
4. Transdermal therapeutic system according to any one of items 1 to 3, wherein the rivastigmine in the rivastigmine-containing layer structure is present in the form of the free base.
5. Transdermal therapeutic system according to any one of items 1 to 4, wherein the amount of rivastigmine contained in the rivastigmine-containing layer structure ranges from 0.5 to 5 mg/cm², preferably from 1 to 3 mg/cm².
6. Transdermal therapeutic system according to any one of items 1 to 5, wherein the rivastigmine-containing layer comprises rivastigmine in an amount of from 20 to 40%, preferably from 25 to 35%, most preferably in an amount of 30% by weight based on the total weight of the rivastigmine-containing layer.
7. Transdermal therapeutic system according to any one of items 1 to 6, wherein the acrylic polymer is an acrylic pressure-sensitive adhesive.
8. Transdermal therapeutic system according to any one of items 1 to 7, wherein the amount of the acrylic polymer ranges from 5 to 40%, preferably from 8 to 35% by weight based on the total weight of the rivastigmine-containing layer.
9. Transdermal therapeutic system according to any one of items 1 to 8, wherein the acrylic polymer is obtainable from one or more monomers selected from acrylic acid, butylacrylate, 2-ethylhexylacrylate, glycidylmethacrylate, 2-hydroxyethylacrylate, methylacrylate, methylmethacrylate, t-octylacrylamide, and vinylacetate, preferably from one or more monomers selected from ethylhexylacrylate, glycidylmethacrylate, 2-hydroxyethylacrylate, and vinylacetate.
10. Transdermal therapeutic system according to any one of items 1 to 9, wherein the acrylic polymer is a COOH-functionalized acrylic polymer, preferably a COOH-functionalized acrylic polymer obtainable from one or more monomers selected from acrylic acid, 2-ethylhexylacrylate, glycidylmethacrylate and methylacrylate, which may be provided as a solution in ethyl acetate and hexane.
11. Transdermal therapeutic system according to any one of items 1 to 10, wherein the rivastigmine-containing layer does not comprise a permeation enhancer or solubilizer.
12. Transdermal therapeutic system according to any one of items 1 to 11, wherein the at least one styrene-isoprene-styrene block copolymer comprises styrene blocks and isoprene blocks in a ratio of from 10:90 (%) to 30:70 (%), preferably in a ratio of 15:85 (%) or 22:78 (%).
13. Transdermal therapeutic system according to any one of items 1 to 12, wherein the at least one styrene-isoprene-styrene block copolymer is obtainable by polymerisation of three blocks of polystyrene, polyisoprene and polystyrene.
14. Transdermal therapeutic system according to any one of items 1 to 13, wherein the at least one tackifier is an alicyclic saturated hydrocarbon resin, or a hydrogenated rosin glycerol ester, or paraffinum liquidum, or a mixture thereof.
15. Transdermal therapeutic system according to any one of items 1 to 14, wherein the at least one tackifier is a mixture comprising an alicyclic saturated hydrocarbon resin and paraffinum liquidum.
16. Transdermal therapeutic system according to any one of items 1 to 15, wherein the at least one tackifier is a mixture comprising a hydrogenated rosin glycerol ester and paraffinum liquidum.
17. Transdermal therapeutic system according to item 14 or 15, wherein the alicyclic saturated hydrocarbon resin is obtainable from polymerisation of an unsaturated hydrocarbon obtainable prepared by the decomposition of petroleum naphtha at elevated temperatures.
18. Transdermal therapeutic system according to item 14 or 16, wherein the hydrogenated rosin glycerol ester is a solid resin obtainable from the hydrogenation of rosin, followed by esterification with glycerin.
19. Transdermal therapeutic system according to any one of items 1 to 18, wherein the amount of tackifier contained in the skin contact layer ranges from 20 to 80%, preferably from 35 to 65%.
20. Transdermal therapeutic system according any one of items 1 to 19, wherein the ratio of the amount of styrene-isoprene-styrene block copolymer(s) to the amount of tackifier(s) is between 60:40 (w/w) to 40:60 (w/w) based on the total weight of the skin contact layer, preferably wherein the ratio of the amount of styrene-isoprene-styrene block copolymer(s) to the amount of tackifier(s) is 50:50 (w/w) based on the total weight of the skin contact layer.
21. Transdermal therapeutic system according to any one of items 1 to 20, wherein the at least one styrene-isoprene-styrene block copolymer and the at least one tackifier are present in the skin contact layer in an overall amount of at least 90% by weight, preferably in an overall amount of at least 99% by weight based on the total weight of the skin contact layer.
22. Transdermal therapeutic system according to any one of items 1 to 21, wherein the area weight of the skin contact layer ranges from 5 to 60 g/m², preferably from 20 to 40 g/m².
23. Transdermal therapeutic system according to any one of items 1 to 22, wherein the area weight of the rivastigmine-containing layer ranges from 40 to 250 g/m², preferably from 50 to 200 g/m².
24. Transdermal therapeutic system according to any one of items 1 to 23, wherein the area of release ranges from 1 to 30 cm², preferably from 2 to 22 cm².
25. Transdermal therapeutic system according to any one of items 1 to 24, wherein the transdermal therapeutic system provides by transdermal delivery a mean release rate of from 150 to 3500 μg/cm²*day, preferably from 400 to 2000 μg/cm²*day rivastigmine over about 24 hours of administration.
26. Transdermal therapeutic system according to any one of items 1 to 25, providing a cumulative permeated amount of rivastigmine as measured in a Franz diffusion cell with an EVA membrane of about 300 to 1200 μg/cm² over a time period of about 24 hours.
27. Transdermal therapeutic system according to any one of items 1 to 26 for use in a method of treating a human patient, preferably for use in a method of preventing, treating, or delaying of progression of Alzheimer's disease, dementia associated with Parkinson's disease, and/or symptoms of traumatic brain injury.
28. Transdermal therapeutic system according to any one of items 1 to 27 for use in a method of treating a human patient, preferably for use in a method of treating mild to moderate dementia caused by Alzheimer's or Parkinson's disease.
29. Transdermal therapeutic system for use according to item 27 or 28, wherein the transdermal therapeutic system is applied to the skin of the patient for a dosing interval of at least 24 hours, preferably about 24 hours.
30. Method of treating a human patient, in particular preventing, treating, or delaying of progression of Alzheimer's disease, dementia associated with Parkinson's disease, and/or symptoms of traumatic brain injury, by applying a transdermal therapeutic system as defined in any one of items 1 to 27 to the skin of the patient.
31. Method of treating a human patient, in particular treating a mild to moderate dementia caused by Alzheimer's and Parkinson's disease, by applying a transdermal therapeutic system as defined in any one of items 1 to 28 to the skin of the patient.
32. Method of treating a human patient according to item 30 or 31, wherein the transdermal therapeutic system is applied to the skin of the patient for a dosing interval of at least 24 hours, preferably about 24 hours.
33. A process for manufacturing a transdermal therapeutic system according to any one of items 1 to 29 comprising the steps of:
   1) providing a rivastigmine-containing coating composition by combining at least the components
      i) rivastigmine; and
      ii) at least one acrylic polymer;
   2) coating the rivastigmine-containing coating composition onto a film in an amount to provide the desired area weight,
   3) drying the coated rivastigmine-containing coating composition to provide the rivastigmine-containing layer,
   4) providing an additional coating composition for an additional skin contact layer by combining at least the components
      a) at least one styrene-isoprene-styrene block copolymer; and
      b) at least one tackifier;
   5) coating and drying the additional coating composition according to steps 2 and 3, wherein the film is a release liner,
   6) laminating the adhesive side of the skin contact layer onto the adhesive side of the rivastigmine-containing layer to provide a rivastigmine-containing layer structure with the desired area of release,
   7) punching the individual systems from the rivastigmine-containing layer structure,
   8) optionally adhering to the individual systems a rivastigmine-free self-adhesive layer structure comprising also a backing layer and a rivastigmine-free pressure-sensitive adhesive layer and which is larger than the individual systems of rivastigmine-containing self-adhesive layer structure.
34. The method of manufacture according to item 33, wherein the film in step 2) is a release liner, wherein the rivastigmine-containing layer is laminated after step 3) to a backing layer, and wherein the release liner of step 2) is removed before step 6).
35. Process for manufacturing a rivastigmine-containing layer according to items 33 or 34, wherein the acrylic polymer is provided as a solution, wherein the solvent is ethyl acetate or n-heptane.
36. Transdermal therapeutic system obtainable by a process in accordance with any one of items 33 to 35.

1. Transdermal therapeutic system for the transdermal administration of rivastigmine comprising a rivastigmine-containing layer structure, said rivastigmine-containing layer structure comprising:
A) a backing layer;
B) a rivastigmine-containing layer comprising at least one acrylic polymer; and
C) a skin contact layer comprising at least one styrene-isoprene-styrene block copolymer and at least one tackifier.

2. Transdermal therapeutic system according to claim 1, wherein the at least one styrene-isoprene-styrene block copolymer comprises styrene blocks and isoprene blocks in a ratio of from 10:90 (%) to 30:70 (%) based on the total weight of the skin contact layer, preferably in a ratio of 15:85 (%) or 22:78 (%) based on the total weight of the skin contact layer.

3. Transdermal therapeutic system according to claim 1 or 2, wherein the at least one tackifier is an alicyclic saturated hydrocarbon resin, or a hydrogenated rosin glycerol ester, or paraffinum liquidum, or a mixture thereof.

4. Transdermal therapeutic system according to any one of claims 1 to 3, wherein the ratio of the amount of styrene-isoprene-styrene block copolymer(s) to the amount of tackifier(s) is between 60:40 (w/w) to 40:60 (w/w), preferably wherein the ratio of the amount of styrene-isoprene-styrene block copolymer(s) to the amount of tackifier(s) is 50:50 (w/w).

5. Transdermal therapeutic system according to any one of claims 1 to 4, wherein the at least one styrene-isoprene-styrene block copolymer and the at least one tackifier are present in the skin contact layer in an overall amount of at least 90% by weight, preferably in an overall amount of at least 99% by weight based on the total weight of the skin contact layer.

6. Transdermal therapeutic system according to any one of claims 1 to 5, wherein the amount of rivastigmine contained in the rivastigmine-containing layer structure ranges from 0.5 to 5 mg/cm², preferably from 1 to 3 mg/cm².

7. Transdermal therapeutic system according to any one of claims 1 to 6, wherein the rivastigmine-containing layer structure comprises rivastigmine in an amount of from 20 to 40%, preferably from 25 to 35%, most preferably in an amount of 30% by weight based on the total area weight of the rivastigmine-containing layer.

8. Transdermal therapeutic system according to any one of claims 1 to 7, wherein the acrylic polymer is a COOH-functionalized acrylic polymer.

9. Transdermal therapeutic system according to any one of claims 1 to 8, wherein the acrylic polymer is obtainable from one or more monomers selected from acrylic acid, 2-ethylhexylacrylate, glycidylmethacrylate and methylacrylate.

10. Transdermal therapeutic system according to any one of claims 1 to 9,
wherein the area weight of the rivastigmine-containing layer ranges from 40 to 250 g/m², preferably from 50 to 200 g/m², and/or
wherein the area of release ranges from 1 to 30 cm², preferably from 2 to 22 cm².

11. Transdermal therapeutic system according to any one of claims 1 to 10, wherein the transdermal therapeutic system provides by transdermal delivery a mean release rate of from 150 to 3500 μg/cm², preferably from 400 to 2000 μg/cm² rivastigmine over about 24 hours of administration.

12. Transdermal therapeutic system according to any one of claims 1 to 11 for use in a method of treating a human patient,
preferably for use in a method of preventing, treating, or delaying of progression of Alzheimer's disease, dementia associated with Parkinson's disease, and/or symptoms of traumatic brain injury, or
for use in a method of treating mild to moderate dementia caused by Alzheimer's or Parkinson's disease.

13. A process for manufacturing a transdermal therapeutic system according to any one of claims 1 to 12 comprising the steps of:
1) providing a rivastigmine-containing coating composition by combining at least the components
i) rivastigmine; and
ii) at least one acrylic polymer;
2) coating the rivastigmine-containing coating composition onto a film in an amount to provide the desired area weight,
3) drying the coated rivastigmine-containing coating composition to provide the rivastigmine-containing layer,
4) providing an additional coating composition for an additional skin contact layer by combining at least the components
a) at least one styrene-isoprene-styrene block copolymer; and
b) at least one tackifier;
5) coating and drying the additional coating composition according to steps 2 and 3, wherein the film is a release liner,
6) laminating the adhesive side of the skin contact layer onto the adhesive side of the rivastigmine-containing layer to provide a rivastigmine-containing layer structure with the desired area of release,
7) punching the individual systems from the rivastigmine-containing layer structure,
8) optionally adhering to the individual systems a rivastigmine-free self-adhesive layer structure comprising also a backing layer and a rivastigmine-free pressure-sensitive adhesive layer and which is larger than the individual systems of rivastigmine-containing self-adhesive layer structure.
